(19) United States
(12) Patent Application Publication
(10) Pub. No.: US 2023/0174642 A1
(43) Pub. Date: Jun. 8, 2023

(54) AFFINITY-MATURATED ANTI-ASIC1a ANTIBODIES

(71) Applicant: SHANGHAITECH UNIVERSITY, Pudong District, Shanghai (CN)

(72) Inventors: Guang YANG, Shanghai (CN); Min QIANG, Shanghai (CN)

(73) Assignee: SHANGHAITECH UNIVERSITY, Pudong District, Shanghai (CN)

(21) Appl. No.: 17/616,492

(22) PCT Filed: Jun. 4, 2019
(86) PCT No.: PCT/CN2019/090041

§ 371 Date: Dec. 3, 2021

(51) Int. Cl. C07K 16/28; G01N 33/68; A61P 25/00

(52) U.S. Cl. CPC C07K 16/28; G01N 33/6872; A61P 25/00; A61K 2039/505

(57) ABSTRACT

Provided are immunoglobulin-related compositions (e.g., antibodies or antigen binding fragments thereof) that specifically bind acid-sensing ion channel 1a (ASIC1a) protein and uses of the same. Also provided is a method of administering an effective amount of the anti-ASIC1a antibodies to treat a subject suffering from, or predisposed to, acidosis, or to treat a subject suffering from a disease caused by or related to altered ASIC1a activity and/or signaling, including ischemic stroke and related conditions.

### TECHNICAL FIELD

The present technology relates generally to the preparation of immunoglobulin-related compositions (e.g., antibodies or antigen binding fragments thereof) that specifically bind acid-sensing ion channel 1a(ASIC1a) protein and uses of the same. More particularly, the present technology relates to administering an effective amount of the anti-ASIC1a antibodies to treat a subject suffering from, or predisposed to, acidosis, or to treat a subject suffering from a disease caused by or related to altered ASIC1a activity and/or signaling, including ischemic stroke and related conditions.

### BACKGROUND

The following description is provided to assist the understanding of the reader. None of the information provided or references cited is admitted to be prior art.

Acid-Sensing Ion Channels (ASICs) are gated by extracellular protons. ASICs are cation channels activated by extracellular acidosis. At least four genes have been identified that encode six ASIC subunits: ASIC1a, ASIC1b, ASIC2a, ASIC2b, ASIC3, and ASIC4, among which, the “a” and “b” designations represent alternatively spliced variants of ASIC1 and ASIC2 genes, ACCN2 and ACCN1, respectively. Functional ASIC channels, which are sensitive to blockade by amiloride, are composed of three subunits assembled in either homomeric or heteromeric forms. ASIC1a is highly expressed in the brain, and forms functional homo-orheteromeric channels with other ASIC isoforms. With an activation threshold near pH7.0, ASIC1a serves as a primary sensor of acidosis in the brain and is implicated in normal as well as patho-physiology.

### SUMMARY OF THE PRESENT DISCLOSURE

In one aspect, the present technology relates to an antibody, or antigen binding fragment thereof comprising a heavy chain immunoglobulin variable domain (V_{H}) and a light chain immunoglobulin variable domain (V_{L}), wherein the V_{H} comprises a V_{H}-CDR1 sequence of SEQ ID NO: 8, a V_{H}—CDR2 sequence of SEQ ID NO: 9, and a V_{H}-CDR3 sequence selected from the group consisting of: SEQ ID NO: 10, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, and SEQ ID NO: 37; and wherein the V_{L} comprises a V_{L}-CDR1 sequence of SEQ ID NO: 3, a V_{L}—CDR2 sequence of SEQ ID NO: 4, and a V_{L}-CDR3 sequence of SEQ ID NO: 5.

Additionally, or alternatively, in some embodiments, the antibody, or antigen binding fragment thereof further comprises a Fe domain of an isotype selected from the group consisting of IgG1, IgG2, IgG3, IgG4, IgA1, IgA2, IgM, IgD, and IgE. Additionally, or alternatively, in some embodiments, the antigen binding fragment is selected from the group consisting of Fab, F(ab′)2, Fab′, scFv, and Fv. Additionally, or alternatively, in some embodiments, the antibody is a monoclonal antibody, a chimeric antibody, a humanized antibody, or a bispecific antibody.

Additionally, or alternatively, in some embodiments, the antibody, or antigen binding fragment thereof binds to ASIC1a. Additionally, or alternatively, in some embodiments, the antibody, or antigen binding fragment thereof is an antagonist of ASIC1a. Additionally, or alternatively, in some embodiments, the antibody, or antigen binding fragment thereof inhibits ASIC1a-mediated, acid-induced currents. Additionally, or alternatively, in some embodiments, the antibody, or antigen binding fragment thereof inhibits ASIC1a-mediated, acid-induced calcium influx.

Additionally, or alternatively the V_{L} comprises SEQ ID NO: 2; and the V_{H} Comprises: a V_{H}-CDR1 sequence of SEQ ID NO: 8, a V_{H}—CDR2 sequence of SEQ ID NO: 9, and a V_{H}-CDR3 sequence of SEQ ID NO: 11; a V_{H}-CDR1 sequence of SEQ ID NO: 8, a V_{H}—CDR2 sequence of SEQ ID NO: 9, and a V_{H}-CDR3 sequence of SEQ ID NO: 13; a V_{H}-CDR1 sequence of SEQ ID NO: 8, a V_{H}—CDR2 sequence of SEQ ID NO: 9, and a V_{H}-CDR3 sequence of SEQ ID NO: 15; a V_{H}-CDR1 sequence of SEQ ID NO: 8, a V_{H}—CDR2 sequence of SEQ ID NO: 9, and a V_{H}-CDR3 sequence of SEQ ID NO: 17; a V_{H}-CDR1 sequence of SEQ ID NO: 8, a V_{H}—CDR2 sequence of SEQ ID NO: 9, and a V_{H}-CDR3 sequence of SEQ ID NO: 19; a V_{H}-CDR1 sequence of SEQ ID NO: 8, a V_{H}—CDR2 sequence of SEQ ID NO: 9, and a V_{H}-CDR3 sequence of SEQ ID NO: 21; a V_{H}-CDR1 sequence of SEQ ID NO: 8, a V_{H}-CDR2 sequence of SEQ ID NO: 9, and a V_{H}-CDR3 sequence of SEQ ID NO: 23; a V_{H}-CDR1 sequence of SEQ ID NO: 8, a V_{H}—CDR2 sequence of SEQ ID NO: 9, and a V_{H}-CDR3 sequence of SEQ ID NO: 25; a V_{H}-CDR1 sequence of SEQ ID NO: 8, a V_{H}—CDR2 sequence of SEQ ID NO: 9, and a V_{H}-CDR3 sequence of SEQ ID NO: 27; a V_{H}-CDR1 sequence of SEQ ID NO: 8, a V_{H}—CDR2 sequence of SEQ ID NO: 9, and a V_{H}-CDR3 sequence of SEQ ID NO:29; a V_{H}-CDR1 sequence of SEQ ID NO: 8, a V_{H}—CDR2 sequence of SEQ ID NO: 9, and a V_{H}-CDR3 sequence of SEQ ID NO: 31; a V_{H}-CDR1 sequence of SEQ ID NO: 8, a V_{H}-CDR2 sequence of SEQ ID NO: 9, and a V_{H}-CDR3 sequence of SEQ ID NO: 33; a V_{H}-CDR1 sequence of SEQ ID NO: 8, a V_{H}—CDR2 sequence of SEQ ID NO: 9, and a V_{H}-CDR3 sequence of SEQ ID NO: 35; or a V_{H}—CDR1 sequence of SEQ ID NO: 8, a V_{H}—CDR2 sequence of SEQ ID NO: 9, and a V_{H}-CDR3 sequence of SEQ ID NO: 37.

Additionally, or alternatively, in some embodiments, the V_{L} comprises a V_{L}-CDR1 sequence of SEQ ID NO: 3, a V_{L}—CDR2 sequence of SEQ ID NO: 4, a V_{L}—CDR3 sequence of SEQ ID NO: 5, and the V_{H} comprises: a V_{H}-CDR1 sequence of SEQ ID NO: 8, a V_{H}—CDR2 sequence of SEQ ID NO: 9, and a V_{H}-CDR3 sequence of SEQ ID NO: 11; a V_{H}-CDR1 sequence of SEQ ID NO: 8, a V_{H}—CDR2 sequence of SEQ ID NO: 9, and a V_{H}-CDR3 sequence of SEQ ID NO: 13; a V_{H}-CDR1 sequence of SEQ ID NO: 8, a V_{H}—CDR2 sequence of SEQ ID NO: 9, and a V_{H}-CDR3 sequence of SEQ ID NO: 15; a V_{H}-CDR1 sequence of SEQ ID NO: 8, a V_{H}—CDR2 sequence of SEQ ID NO: 9, and a V_{H}-CDR3 sequence of SEQ ID NO: 17; a V_{H}-CDR1 sequence of SEQ ID NO: 8, a V_{H}—CDR2 sequence of SEQ ID NO: 9, and a V_{H}-CDR3 sequence of SEQ ID NO: 19; a V_{H}-CDR1 sequence of SEQ ID NO: 8, a V_{H}-CDR2 sequence of SEQ ID NO: 9, and a V_{H}-CDR3 sequence of SEQ ID NO: 21; a V_{H}-CDR1 sequence of SEQ ID NO: 8, a V_{H}—CDR2 sequence of SEQ ID NO: 9, and a V_{H}-CDR3 sequence of SEQ ID NO: 23; a V_{H}-CDR1 sequence of SEQ ID NO: 8, a V_{H}—CDR2 sequence of SEQ ID NO: 9, and a V_{H}-CDR3 sequence of SEQ ID NO: 25; a V_{H}-CDR1 sequence of SEQ ID NO: 8, a V_{H}—CDR2 sequence of SEQ ID NO: 9, and a V_{H}-CDR3 sequence of SEQ ID NO:27; a V_{H}-CDR1 sequence of SEQ ID NO: 8, a V_{H}—CDR2 sequence of SEQ ID NO: 9, and a V_{H}-CDR3 sequence of SEQ ID NO:29; a V_{H}-CDR1 sequence of SEQ ID NO: 8, a V_{H}-CDR2 sequence of SEQ ID NO: 9, and a V_{H}-CDR3 sequence of SEQ ID NO: 31; a V_{H}-CDR1 sequence of SEQ ID NO: 8, a V_{H}—CDR2 sequence of SEQ ID NO: 9, and a V_{H}-CDR3 sequence of SEQ ID NO:33; a V_{H}-CDR1 sequence of SEQ ID NO: 8, a V_{H}—CDR2 sequence of SEQ ID NO: 9, and a V_{H}-CDR3 sequence of SEQ ID NO: 35; or a V_{H}—CDR1 sequence of SEQ ID NO: 8, a V_{H}—CDR2 sequence of SEQ ID NO: 9, and a V_{H}-CDR3 sequence of SEQ ID NO: 37.

In one aspect, the present disclosure provides an antibody, or antigen binding fragment thereof comprising (a) a light chain immunoglobulin variable domain sequence (V_{L}) that is at least 80%, at least 85%, at least 90%, at least 95%, or at least 99% identical to the light chain immunoglobulin variable domain sequence present in SEQ ID NO: 2; and/or (b) a heavy chain immunoglobulin variable domain sequence (V_{H}) comprising (b1) a V_{H}-CDR1 that is at least 80%, at least 85%, at least 90%, at least 95%, or at least 99% identical to the V_{H}-CDR1 present in SEQ ID NO: 8; (b2) a V_{H}-CDR2 that is at least 80%, at least 85%, at least 90%, at least 95%, or at least 99% identical to the V_{H}-CDR2 present in SEQ ID NO: 9, and/or (b3) a V_{H}-CDR3 that is at least 80%, at least 85%, at least 90%, at least 95%, or at least 99% identical to the V_{H}-CDR3 present in any one of SEQ ID NO: 10, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, or SEQ ID NO: 37.

Additionally, or alternatively, in some embodiments, the V_{L} Comprises a V_{L}-CDR1 that is at least 80%, at least 85%, at least 90%, at least 95%, or at least 99% identical to the V_{H}-CDR1 present in SEQ ID NO: 3; a V_{L}-CDR2 that is at least 80%, at least 85%, at least 90%, at least 95%, or at least 99% identical to the V_{H}-CDR2 present in SEQ ID NO: 4; and/or a V_{L}—CDR3 that is at least 80%, at least 85%, at least 90%, at least 95%, or at least 99% identical to the V_{H}-CDR3 present in SEQ ID NO: 5.

In one aspect, the present technology relates to an antibody, or antigen binding fragment thereof comprising a heavy chain immunoglobulin variable domain (V_{H}) and a light chain immunoglobulin variable domain (V_{L}), wherein the V_{H} Comprises an amino acid sequence of SEQ ID NO:7 and the V_{L} comprises an amino acid sequence of SEQ ID NO: 2.

In one aspect, the present disclosure provides an antibody, or antigen binding fragment thereof comprising (a) a light chain immunoglobulin variable domain sequence (V_{L}) that is at least 80%, at least 85%, at least 90%, at least 95%, or at least 99% identical to the light chain immunoglobulin variable domain sequence of SEQ ID NO: 2; and/or (b) a heavy chain immunoglobulin variable domain sequence (V_{H}) that is at least 80%, at least 85%, at least 90%, at least 95%, or at least 99% identical to the heavy chain immunoglobulin variable domain sequence of SEQ ID NO: 7.

In one aspect, the present technology relates to an antibody, or antigen binding fragment thereof comprising antibody, or antigen binding fragment thereof, comprising a light chain (LC) and a heavy chain (HC), wherein the LC comprises an amino acid sequence comprising SEQ ID NO: 2, and wherein HC comprises a heavy chain immunoglobulin variable domain (V_{H}), wherein the V_{H} comprises a V_{H}-CDR1 sequence of SEQ ID NO: 8, a V_{H}—CDR2 sequence of SEQ ID NO: 9, and a V_{H}-CDR3 sequence selected from the group consisting of: SEQ ID NO: 10, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, and SEQ ID NO: 37.

In one aspect, the present technology relates to an antibody, or antigen binding fragment thereof comprising a heavy chain immunoglobulin variable domain (V_{H}) and a light chain immunoglobulin variable domain (V_{L}), wherein the V_{L} comprises an amino acid sequence of SEQ ID NO: 2. Additionally, or alternatively, in some embodiments, the V_{H} comprises: a V_{H}-CDR1 sequence of SEQ ID NO: 8, a V_{H}—CDR2 sequence of SEQ ID NO: 9, and a V_{H}-CDR3 sequence of SEQ ID NO: 11; a V_{H}-CDR1 sequence of SEQ ID NO: 8, a V_{H}-CDR2 sequence of SEQ ID NO: 9, and a V_{H}-CDR3 sequence of SEQ ID NO: 13; a V_{H}-CDR1 sequence of SEQ ID NO: 8, a V_{H}—CDR2 sequence of SEQ ID NO: 9, and a V_{H}-CDR3 sequence of SEQ ID NO: 15; a V_{H}-CDR1 sequence of SEQ ID NO: 8, a V_{H}—CDR2 sequence of SEQ ID NO: 9, and a V_{H}-CDR3 sequence of SEQ ID NO: 17; a V_{H}-CDR1 sequence of SEQ ID NO: 8, a V_{H}—CDR2 sequence of SEQ ID NO: 9, and a V_{H}-CDR3 sequence ofSEQ ID NO: 19; a V_{H}-CDR1 sequence of SEQ ID NO: 8, a V_{H}—CDR2 sequence of SEQ ID NO: 9, and a V_{H}-CDR3 sequence of SEQ ID NO: 21; a V_{H}-CDR1 sequence of SEQ ID NO: 8, a V_{H}-CDR2 sequence of SEQ ID NO: 9, and a V_{H}-CDR3 sequence of SEQ ID NO: 23; a V_{H}-CDR1 sequence of SEQ ID NO: 8, a V_{H}—CDR2 sequence of SEQ ID NO: 9, and a V_{H}-CDR3 sequence of SEQ ID NO: 25; a V_{H}-CDR1 sequence of SEQ ID NO: 8, a V_{H}—CDR2 sequence of SEQ ID NO: 9, and a V_{H}-CDR3 sequence of SEQ ID NO:27; a V_{H}-CDR1 sequence of SEQ ID NO: 8, a V_{H}—CDR2 sequence of SEQ ID NO: 9, and a V_{H}-CDR3 sequence of SEQ ID NO:29; a V_{H}-CDR1 sequence of SEQ ID NO: 8, a V_{H}—CDR2 sequence of SEQ ID NO: 9, and a V_{H}-CDR3 sequence of SEQ ID NO: 31; a V_{H}-CDR1 sequence of SEQ ID NO: 8, a V_{H}-CDR2 sequence of SEQ ID NO: 9, and a V_{H}-CDR3 sequence of SEQ ID NO:33; a V_{H}-CDR1 sequence of SEQ ID NO: 8, a V_{H}—CDR2 sequence of SEQ ID NO: 9, and a V_{H}-CDR3 sequence of SEQ ID NO: 35; or a V_{H}—CDR1 sequence of SEQ ID NO: 8, a V_{H}—CDR2 sequence of SEQ ID NO: 9, and a V_{H}-CDR3 sequence of SEQ ID NO: 37.

In one aspect, the present technology relates to a method of treating acidosis in a subject in need thereof, comprising administering a therapeutically effective amount of an effective amount of an antibody or antigen binding fragment thereof comprising a heavy chain immunoglobulin variable domain (V_{H}) and a light chain immunoglobulin variable domain (V_{L}), wherein the V_{H} comprises a V_{H}-CDR1 sequence of SEQ ID NO: 8, a V_{H}—CDR2 sequence of SEQ ID NO: 9, and a V_{H}-CDR3 sequence selected from the group consisting of: SEQ ID NO: 10, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, and SEQ ID NO: 37; and wherein the V_{L} comprises a V_{L}-CDR1 sequence of SEQ ID NO: 3, a V_{L}—CDR2 sequence of SEQ ID NO: 4, and a V_{L}-CDR3 sequence of SEQ ID NO: 5. Additionally, or alternatively, in some embodiments, the V_{H}-CDR3 sequence selected from the group consisting of: SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, and SEQ ID NO: 37.

In one aspect, the present technology relates to a treating ischemic stroke in a subject in need thereof, comprising administering a therapeutically effective amount of an effective amount of an antibody or antigen binding fragment thereof comprising a heavy chain immunoglobulin variable domain (V_{H}) and a light chain immunoglobulin variable domain (V_{L}), wherein the V_{H} comprises a V_{H}-CDR1 sequence of SEQ ID NO: 8, a V_{H}—CDR2 sequence of SEQ ID NO: 9, and a V_{H}-CDR3 sequence selected from the group consisting of: SEQ ID NO: 10, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, and SEQ ID NO: 37; and wherein the V_{L} comprises a V_{L}-CDR1 sequence of SEQ ID NO: 3, a V_{L}—CDR2 sequence of SEQ ID NO: 4, and a V_{L}-CDR3 sequence of SEQ ID NO: 5. Additionally, or alternatively, in some embodiments, the V_{H}-CDR3 sequence selected from the group consisting of: SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, and SEQ ID NO: 37.

In one aspect, the present technology relates to a method of treating a disorder caused by or related to ASIC1a activity and/or signaling in a subject in need thereof, comprising administering a therapeutically effective amount of an effective amount of an antibody or antigen binding fragment thereof a heavy chain immunoglobulin variable domain (V_{H}) and a light chain immunoglobulin variable domain (V_{L}), wherein the V_{H} comprises a V_{H}-CDR1 sequence of SEQ ID NO: 8, a V_{H}—CDR2 sequence of SEQ ID NO: 9, and a V_{H}-CDR3 sequence selected from the group consisting of: SEQ ID NO: 10, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, and SEQ ID NO: 37; and wherein the V_{L} comprises a V_{L}-CDR1 sequence of SEQ ID NO: 3, a V_{L}—CDR2 sequence of SEQ ID NO: 4, and a V_{L}-CDR3 sequence of SEQ ID NO: 5. Additionally, or alternatively, in some embodiments, the V_{H}-CDR3 sequence selected from the group consisting of: SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, and SEQ ID NO: 37. Additionally, or alternatively, in some embodiments, the disorder caused by or related to ASIC1a activity and/or signaling is a neurodegenerative disease, neuropsychological disease, epilepsy, multiple sclerosis, pain and migraine.

In one aspect, the present technology provides a nucleic acid sequence encoding any of the immunoglobulin-related compositions described herein. Also disclosed herein are recombinant nucleic acid sequences encoding any of the antibodies described herein. In some embodiments, the nucleic acid sequence is selected from the group consisting of SEQ ID NOs: 1, 6, 12, 14, 16, 18, 20, 22, 24, 26, 28, 30, 32, 34, 36 and 38.

In another aspect, the present technology provides a host cell or a vector expressing any nucleic acid sequence encoding any of the immunoglobulin-related compositions described herein.

In another aspect, the present technology provides a kit comprising the antibody, or antigen binding fragment thereof of any of the embodiments disclosed herein and instructions for use. In some embodiments, the antibody, or antigen binding fragment thereof of any of the embodiments disclosed herein is coupled to at least one detectable label selected from the group consisting of a radioactive label, a fluorescent label, and a chromogenic label. In some embodiments, the kit further comprises a secondary antibody that specifically binds to the antibody, or antigen binding fragment thereof of any of the embodiments disclosed herein.

In another aspect, the present technology provides a method for detecting ASIC1a in a biological sample comprising contacting the biological sample with the antibody, or antigen binding fragment thereof of any of the embodiments disclosed herein, conjugated to a detectable label; and detecting the presence and the level of the detectable label in the biological sample.

### BRIEF DESCRIPTION OF THE DRAWINGS

FIG. 1(R1 to R5) shows the results of FACS sorting during five successive rounds of selection of a yeast display library for ASIC1a binders. Shown at the bottom right is a consensus sequence derived from sequence alignment of V_{H}-CDR3 sequences that were selected after the fifth round of selection.

FIG. 2 shows the binding of the affinity-matured derivatives of the ASC06-IgG1 antibody to CHO-K1 cells expressing human ASIC1a-eYFP (hASIC1a-eYFP), mouse ASIC1a-eYFP (mASIC1a-eYFP), or rat-ASIC1a-eYFP(rASIC1a-eYFP) as measured by FACS. The CHO-K1 cells, which were transiently transfected with plasmids encoding hASIC1a-eYFP, mASIC1a-eYFP, or rASIC1a-eYFP, were stained with affinity-matured versions of ASC06-IgG1 (red), and subjected to FACS analysis.

FIG. 3 demonstrates the subtype specificity of the affinity-matured antibodies. The CHO-K1 cells, which were transiently transfected with plasmids encoding human ASIC1b(hASIC1b)-eYFP, human ASIC2a(hASIC2a)-eYFP and human ASIC3a(hASIC3a)-eYFP, were stained with ASC06-IgG1 or its affinity-matured versions, and subjected to FACS sorting. The absence of double-positive cell populations expressing eYFP and Alexa555 fluorescence (in the upper right quadrant of the FACS profiles) suggested the lack of binding of ASC06-IgG1 or affinity-matured versions to the hASIC1b, hASIC2a or hASIC3a subtypes of cell surface ASICs. Each plot displays 10,000 cells.

FIGS. 4A-4E show the effect of affinity-matured ASC06-IgG1 derivative antibodies on the hASIC1a currents. Shown are the representative current traces recorded from single CHO-K1 cells stably expressing hASIC1a in the absence or presence of 100 nM ASC06-IgG1 (FIG. 4A) or affinity-matured ASC06-IgG1 versions ASC06-01-IgG1 (FIG. 4B), ASC06-02-IgG1 (FIG. 4C), ASC06-03-IgG1 (FIG. 4D), or ASC06-04-IgG1 (FIG. 4E). Amiloride (30 μM) was used as a positive control. “Wash” represents the recovery of the current after the treatment of 100 nM indicated antibody followed by 15 min infusion of washing solution.

FIG. 5 shows the effect of increasing doses of ASC06-IgG1 on the acid-induced ASIC1a currents as measured by the fluorescent membrane potential (FMP) assay (n=6).

FIG. 6 shows the effect of increasing doses of ASC06-IgG1 on the ASIC1a-mediated calcium influx as measured by a Fluorescent Imaging Plate Reader (FLIPR) based assay (n=6).

FIG. 7A shows the nucleotide sequence of V_{L} of ASC06 (SEQ ID NO: 1).

FIG. 7B shows the amino acid sequence of V_{L} of ASC06 (SEQ ID NO: 2). V_{L}—CDR1 (SEQ ID NO: 3), V_{L}—CDR2 (SEQ ID NO: 4) and V_{L}—CDR3 (SEQ ID NO: 5) are indicated by an underlined boldface font.

FIG. 7C shows the nucleotide sequence of V_{H} of ASC06 (SEQ ID NO: 6).

FIG. 7D shows the amino acid sequence of V_{H} of ASC06 (SEQ ID NO: 7). V_{H}-CDR1 (SEQ ID NO: 8), V_{H}—CDR2 (SEQ ID NO: 9) and V_{H}—CDR3 (SEQ ID NO: 10) are indicated by an underlined boldface font.

### DETAILED DESCRIPTION

It is to be appreciated that certain aspects, modes, embodiments, variations and features of the present technology are described below in various levels of detail in order to provide a substantial understanding of the present technology. The present technology provides methods of treating ischemic stroke and/or related disorders.

While the exemplified antibodies that target the ASIC1a protein described herein are scFv and IgG1 antibodies, the description is intended to embrace broadly to any immunologic binding agent, such as IgG, IgM, IgA, IgD, IgE, and genetically modified IgG, and fragments thereof as well as polypeptides comprising antibody complementarity determining regions (CDR) domains that retain the antigen binding activity described herein.

### Definitions

The definitions of certain terms as used in this specification are provided below. Unless defined otherwise, all technical and scientific terms used herein generally have the same meaning as commonly understood by one of ordinary skill in the art to which this present technology belongs.

As used in this specification and the appended claims, the singular forms “a”, “an” and “the” include plural referents unless the content clearly dictates otherwise. For example, reference to “a cell” includes a combination of two or more cells, and the like. Generally, the nomenclature used herein and the laboratory procedures in cell culture, molecular genetics, organic chemistry, analytical chemistry and nucleic acid chemistry and hybridization described below are those well-known and commonly employed in the art.

As used herein, the term “about” in reference to a number is generally taken to include numbers that fall within a range of 1%, 5%, or 10% in either direction (greater than or less than) of the number unless otherwise stated or otherwise evident from the context (except where such number would be less than 0% or exceed 100% of a possible value).

As used herein, the term “acidosis” or “acidemia” is used to refer to a condition associated with increased acidity in the blood and other body tissues, and a state of low blood pH and/or tissue pH. Acidosis or acidemia occurs when arterial pH falls below 7.35, except in fetuses. Fetal acidosis acidemia is defined as an umbilical vessel pH of less than 7.20. Metabolic acidosis may result from either increased production of metabolic acids, such as lactic acid, which is produced during anaerobic metabolism, or disturbances in the ability to excrete acid via the kidneys. Respiratory acidosis results from a build-up of carbon dioxide in the blood (hypercapnia) due to hypoventilation. Signs and symptoms that may be seen in acidosis include headaches, confusion, feeling tired, tremors, sleepiness, flapping tremor, and dysfunction of the cerebrum of the brain which may progress to coma.

As used herein, the “administration” of an agent, drug, or peptide to a subject includes any route of introducing or delivering to a subject a compound to perform its intended function. Administration can be carried out by any suitable route, including orally, intranasally, parenterally (intravenously, intramuscularly, intraperitoneally, or subcutaneously), or topically. In some embodiments, the anti-ASIC1a antibodies of the present technology is administered by an intracranial route or an intra-arterial route. Administration includes self-administration and the administration by another.

As used herein, the term “amino acid” is used to refer to any organic molecule that contains at least one amino group and at least one carboxyl group. Typically, at least one amino group is at the a position relative to a carboxyl group. The term “amino acid” includes naturally-occurring amino acids and synthetic amino acids, as well as amino acid analogs and amino acid mimetics that function in a manner similar to the naturally-occurring amino acids. Naturally-occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally-occurring amino acid, i.e., an α-carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally-occurring amino acid. Amino acid mimetics refer to chemical compounds that have a structure that is different from the general chemical structure of an amino acid, but that functions in a manner similar to a naturally-occurring amino acid. Amino acids can be referred to herein by either their commonly known three letter symbols or by the one-letter symbols recommended by the IUPAC-IUB Biochemical Nomenclature Commission.

As used herein, the term “antibody” collectively refers to immunoglobulins or immunoglobulin-like molecules including by way of example and without limitation, IgA, IgD, IgE, IgG and IgM, combinations thereof, and similar molecules produced during an immune response in any vertebrate, for example, in mammals such as humans, goats, rabbits and mice, as well as non-mammalian species, such as shark immunoglobulins. As used herein, “antibodies” (includes intact immunoglobulins) and “antigen binding fragments” specifically bind to a molecule of interest (or a group of highly similar molecules of interest) to the substantial exclusion of binding to other molecules (for example, antibodies and antibody fragments that have a binding constant for the molecule of interest that is at least 10³ M⁻¹ greater, at least 10⁴ M⁻¹ greater or at least 10⁵ M⁻¹ greater than a binding constant for other molecules in a biological sample). The term “antibody” also includes genetically engineered forms such as chimeric antibodies (for example, humanized murine antibodies), heteroconjugate antibodies (such as, bispecific antibodies). See also, Pierce Catalog and Handbook, 1994-1995 (Pierce Chemical Co., Rockford, Ill.); Kuby, J., Immunology, 3^{rd} Ed., W. H. Freeman & Co., New York, 1997.

More particularly, antibody refers to a polypeptide ligand comprising at least a light chain immunoglobulin variable region or heavy chain immunoglobulin variable region which specifically recognizes and binds an epitope of an antigen. Antibodies are composed of a heavy and a light chain, each of which has a variable region, termed the variable heavy (V_{H}) region and the variable light (V_{L}) region. Together, the V_{H} region and the V_{L} region are responsible for binding the antigen recognized by the antibody. Typically, an immunoglobulin has heavy (H) chains and light (L) chains interconnected by disulfide bonds. There are two types of light chain, lambda (λ) and kappa (κ). There are five main heavy chain classes (or isotypes) which determine the functional activity of an antibody molecule: IgM, IgD, IgG, IgA and IgE. Each heavy and light chain contains a constant region and a variable region, (the regions are also known as “domains”). In combination, the heavy and the light chain variable regions specifically bind the antigen. Light and heavy chain variable regions contain a “framework” region interrupted by three hypervariable regions, also called “complementarity-determining regions” or “CDRs”. The extent of the framework region and CDRs have been defined (see, Kabat et al., Sequences of Proteins of Immunological Interest, U.S. Department of Health and Human Services, 1991, which is hereby incorporated by reference). The Kabat database is now maintained online. The sequences of the framework regions of different light or heavy chains are relatively conserved within a species. The framework region of an antibody, that is the combined framework regions of the constituent light and heavy chains, largely adopt a p-sheet conformation and the CDRs form loops which connect, and in some cases form part of, the 3-sheet structure. Thus, framework regions act to form a scaffold that provides for positioning the CDRs in correct orientation by inter-chain, non-covalent interactions.

The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, a V_{H}—CDR3 is located in the variable domain of the heavy chain of the antibody in which it is found, whereas a V_{L}-CDR1 is the CDR1 from the variable domain of the light chain of the antibody in which it is found. An antibody that binds ASIC1a protein will have a specific V_{H} region and the V_{L} region sequence, and thus specific CDR sequences. Antibodies with different specificities (i.e. different combining sites for different antigens) have different CDRs. Although it is the CDRs that vary from antibody to antibody, only a limited number of amino acid positions within the CDRs are directly involved in antigen binding. These positions within the CDRs are called specificity determining residues (SDRs). “Anti-ASIC1a antibodies of the present technology” as used herein, refers to antibodies (including monoclonal antibodies, polyclonal antibodies, humanized antibodies, chimeric antibodies, recombinant antibodies, multispecific antibodies, bispecific antibodies, etc.) as well as antibody fragments. An antibody or antigen binding fragment thereof specifically binds to an antigen.

As used herein, the term “antibody-related polypeptide” means antigen-binding antibody fragments, including single-chain antibodies, that can comprise the variable region(s) alone, or in combination, with all or part of the following polypeptide elements: hinge region, CH₁, CH₂, and CH₃ domains of an antibody molecule. Also included in the technology are any combinations of variable region(s) and hinge region, CH₁, CH₂, and CH₃ domains. Antibody-related molecules useful in the present methods, e.g., but are not limited to, Fab, Fab′ and F(ab′)₂, Fd, single-chain Fvs (scFv), single-chain antibodies, disulfide-linked Fvs (sdFv) and fragments comprising either a V_{L} or V_{H} domain. Examples include: (i) a Fab fragment, a monovalent fragment consisting of the V_{L}, V_{H}, C_{L} and CH₁ domains; (ii) a F(ab′)₂ fragment, a bivalent fragment comprising two Fab fragments linked by a disulfide bridge at the hinge region; (iii) a Fd fragment consisting of the V_{H} and CH₁ domains; (iv) a Fv fragment consisting of the V_{L} and V_{H} domains of a single arm of an antibody, (v) a dAb fragment (Ward et al., Nature 341: 544-546, 1989), which consists of a V_{H} domain; and (vi) an isolated complementarity determining region (CDR). As such “antibody fragments” or “antigen binding fragments” can comprise a portion of a full length antibody, generally the antigen binding or variable region thereof. Examples of antibody fragments or antigen binding fragments include Fab, Fab′, F(ab′)₂, and Fv fragments; diabodies; linear antibodies; single-chain antibody molecules; and multispecific antibodies formed from antibody fragments.

As used herein, the term “conjugated” refers to the association of two molecules by any method known to those in the art. Suitable types of associations include chemical bonds and physical bonds. Chemical bonds include, for example, covalent bonds and coordinate bonds. Physical bonds include, for instance, hydrogen bonds, dipolar interactions, van der Waal forces, electrostatic interactions, hydrophobic interactions and aromatic stacking.

As used herein, the term “diabodies” refers to small antibody fragments with two antigen-binding sites, which fragments comprise a heavy-chain variable domain (V_{H}) connected to a light-chain variable domain (V_{L}) in the same polypeptide chain (V_{H} V_{L}). By using a linker that is too short to allow pairing between the two domains on the same chain, the domains are forced to pair with the complementary domains of another chain and create two antigen binding sites. Diabodies are described more fully in, e.g., EP 404,097; WO 93/11161; and Hollinger et al., Proc. Natl. Acad. Sci. USA, 90: 6444-6448 (1993).

As used herein, the terms “single-chain antibodies” or “single-chain Fv (scFv)” refer to an antibody fusion molecule of the two domains of the Fv fragment, V_{L} and V_{H}. Single-chain antibody molecules may comprise a polymer with a number of individual molecules, for example, dimer, trimer or other polymers. Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single-chain Fv (scFv)). Bird et al. (1988) Science 242:423-426 and Huston et al. (1988) Proc. Natd. Acad Sci. USA 85:5879-5883. Such single-chain antibodies can be prepared by recombinant techniques or enzymatic or chemical cleavage of intact antibodies.

As used herein, an “antigen” refers to a molecule to which an antibody (or antigen binding fragment thereof) can selectively bind. The target antigen may be a protein, carbohydrate, nucleic acid, lipid, hapten, or other naturally occurring or synthetic compound. In some embodiments, the target antigen may be a polypeptide (e.g., a ASIC1a polypeptide). An antigen may also be administered to an animal to generate an immune response in the animal.

The term “antigen binding fragment” refers to a fragment of the whole immunoglobulin structure which possesses a part of a polypeptide responsible for binding to antigen. Examples of the antigen binding fragment useful in the present technology include scFv, (scFv)₂, Fab, Fab′ and F(ab′)₂, but are not limited thereto.

Any of the above-noted antibody fragments are obtained using conventional techniques known to those of skill in the art, and the fragments are screened for binding specificity and neutralization activity in the same manner as are intact antibodies.

By “binding affinity” is meant the strength of the total noncovalent interactions between a single binding site of a molecule (e.g., an antibody) and its binding partner (e.g., an antigen or antigenic peptide). The affinity of a molecule X for its partner Y can generally be represented by the dissociation constant (K_{D}). Affinity can be measured by standard methods known in the art, including those described herein. A low-affinity complex contains an antibody that generally tends to dissociate readily from the antigen, whereas a high-affinity complex contains an antibody that generally tends to remain bound to the antigen for a longer duration.

As used herein, the term “biological sample” means sample material derived from living cells. Biological samples may include tissues, cells, protein or membrane extracts of cells, and biological fluids (e.g., ascites fluid or cerebrospinal fluid (CSF)) isolated from a subject, as well as tissues, cells and fluids present within a subject. Biological samples of the present technology include, but are not limited to, samples taken from breast tissue, renal tissue, the uterine cervix, the endometrium, the head or neck, the gallbladder, parotid tissue, the prostate, the brain, the pituitary gland, kidney tissue, muscle, the esophagus, the stomach, the small intestine, the colon, the liver, the spleen, the pancreas, thyroid tissue, heart tissue, lung tissue, the bladder, adipose tissue, lymph node tissue, the uterus, ovarian tissue, adrenal tissue, testis tissue, the tonsils, thymus, blood, hair, buccal, skin, serum, plasma, CSF, semen, prostate fluid, seminal fluid, urine, feces, sweat, saliva, sputum, mucus, bone marrow, lymph, and tears. Biological samples can also be obtained from biopsies of internal organs or from cancers. Biological samples can be obtained from subjects for diagnosis or research or can be obtained from non-diseased individuals, as controls or for basic research. Samples may be obtained by standard methods including, e.g., venous puncture and surgical biopsy. In certain embodiments, the biological sample is a skin tissue, hair, nails, sebaceous glands, or a muscle biopsy sample.

As used herein, a “control” is an alternative sample used in an experiment for comparison purpose. A control can be “positive” or “negative.” For example, where the purpose of the experiment is to determine a correlation of the efficacy of a therapeutic agent for the treatment for a particular type of disease, a positive control (a compound or composition known to exhibit the desired therapeutic effect) and a negative control (a subject or a sample that does not receive the therapy or receives a placebo) are typically employed.

As used herein, the term “effective amount” refers to a quantity sufficient to achieve a desired therapeutic and/or prophylactic effect, e.g., an amount which results in the prevention of, or a decrease in a disease or condition described herein or one or more signs or symptoms associated with a disease or condition described herein. In the context of therapeutic or prophylactic applications, the amount of a composition administered to the subject will vary depending on the composition, the degree, type, and severity of the disease and on the characteristics of the individual, such as general health, age, sex, body weight and tolerance to drugs. The skilled artisan will be able to determine appropriate dosages depending on these and other factors. The compositions can also be administered in combination with one or more additional therapeutic compounds. In the methods described herein, the therapeutic compositions may be administered to a subject having one or more signs or symptoms of a disease or condition described herein. As used herein, a “therapeutically effective amount” of a composition refers to composition levels in which the physiological effects of a disease or condition are ameliorated or eliminated. A therapeutically effective amount can be given in one or more administrations.

An “isolated” or “purified” polypeptide or peptide is substantially free of cellular material or other contaminating polypeptides from the cell or tissue source from which the agent is derived, or substantially free from chemical precursors or other chemicals when chemically synthesized. For example, isolated anti-ASIC1a antibodies of the present technology would be free of materials that would interfere with diagnostic or therapeutic uses of the agent. Such interfering materials may include enzymes, hormones and other proteinaceous and nonproteinaceous solutes.

As used herein, the term “epitope” means a protein determinant capable of specific binding to an antibody. Epitopes usually consist of chemically active surface groupings of molecules such as amino acids or sugar side chains and usually have specific three dimensional structural characteristics, as well as specific charge characteristics. Conformational and non-conformational epitopes are distinguished in that the binding to the former but not the latter is lost in the presence of denaturing solvents. In some embodiments, an “epitope” is a region of the ASIC1a protein trimer to which the anti-ASIC1a antibodies of the present technology specifically bind, including extracellular domain of the ASIC1a. In some embodiments, the epitope may span two ASIC1a monomers. In some embodiments, the epitope is a conformational epitope or a non-conformational epitope. To screen for anti-ASIC1a antibodies which bind to an epitope, a routine cross-blocking assay such as that described in Antibodies, A Laboratory Manual, Cold Spring Harbor Laboratory, Ed Harlow and David Lane (1988), can be performed. This assay can be used to determine if an anti-ASIC1a antibody binds the same site or epitope as an anti-ASIC1a antibody of the present technology. Alternatively, or additionally, epitope mapping can be performed by methods known in the art. For example, the antibody sequence can be mutagenized such as by alanine scanning, to identify contact residues. In a different method, peptides corresponding to different regions of ASIC1a protein can be used in competition assays with the test antibodies or with a test antibody and an antibody with a characterized or known epitope.

As used herein, “expression” includes one or more of the following: transcription of the gene into precursor mRNA; splicing and other processing of the precursor mRNA to produce mature mRNA; mRNA stability; translation of the mature mRNA into protein (including codon usage and tRNA availability); and glycosylation and/or other modifications of the translation product, if required for proper expression and function.

As used herein, the term “gene” means a segment of DNA that contains all the information for the regulated biosynthesis of an RNA product, including promoters, exons, introns, and other untranslated regions that control expression.

As used herein, the terms “Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. A polynucleotide or polynucleotide region (or a polypeptide or polypeptide region) has a certain percentage (for example, at least 60%, 65%, 70%, 75%, 80%, 85%, 90%, 95%, 98% or 99%) of “sequence identity” to another sequence means that, when aligned, that percentage of bases (or amino acids) are the same in comparing the two sequences. This alignment and the percent homology or sequence identity can be determined using software programs known in the art. In some embodiments, default parameters are used for alignment.

One alignment program is BLAST, using default parameters. In particular, programs are BLASTN and BLASTP, using the following default parameters: Genetic code=standard; filter=none; strand=both; cutoff=60; expect=10; Matrix=BLOSUM62; Descriptions=50 sequences; [symbol] HIGH by SCORE; Databases=non-redundant, GenBank+EMBL+DDBJ+PDB+GenBank CDS translations+SwissProtein+SPupdate+PIR. Details of these programs can be found at the National Center for Biotechnology Information. Biologically equivalent polynucleotides are those having the specified percent homology and encoding a polypeptide having the same or similar biological activity. Two sequences are deemed “unrelated” or “non-homologous” if they share less than 40% identity, or less than 25% identity, with each other.

As used herein, “humanized” forms of non-human (e.g., murine) antibodies are chimeric antibodies which contain minimal sequence derived from non-human immunoglobulin. For the most part, humanized antibodies are human immunoglobulins in which hypervariable region residues of the recipient are replaced by hypervariable region residues from a non-human species (donor antibody) such as mouse, rat, rabbit or nonhuman primate having the desired specificity, affinity, and capacity. In some embodiments, Fv framework region (FR) residues of the human immunoglobulin are replaced by corresponding non-human residues. Furthermore, humanized antibodies may comprise residues which are not found in the recipient antibody or in the donor antibody. These modifications are made to further refine antibody performance such as binding affinity. Generally, the humanized antibody will comprise substantially all of at least one, and typically two, variable domains (e.g., Fab, Fab′, F(ab′)₂, or Fv), in which all or substantially all of the hypervariable loops correspond to those of a non-human immunoglobulin and all or substantially all of the FR regions are those of a human immunoglobulin consensus FR sequence although the FR regions may include one or more amino acid substitutions that improve binding affinity. The number of these amino acid substitutions in the FR are typically no more than 6 in the H chain, and in the L chain, no more than 3. The humanized antibody optionally may also comprise at least a portion of an immunoglobulin constant region (Fc), typically that of a human immunoglobulin. For further details, see Jones et al., Nature 321:522-525 (1986); Riechmann et al., Nature 332: 323-327 (1988); and Presta, Curr. Op. Struct. Biol. 2:593-596 (1992). See e.g., Ahmed & Cheung, FEBS Letters 588(2):288-297 (2014); Saxena & Wu, Frontiers in immunology 7: 580 (2016).

As used herein, the terms “identical” or percent “identity”, when used in the context of two or more nucleic acids or polypeptide sequences, refer to two or more sequences or subsequences that are the same or have a specified percentage of amino acid residues or nucleotides that are the same (i.e., about 60%, 65%, 70%, 75%, 80%, 85%, 90%, 91%, 92%, 93%, 94%, 95%, 96%, 97%, 98%, 99%, or higher identity over a specified region (e.g., nucleotide sequence encoding an antibody described herein or amino acid sequence of an antibody described herein)), when compared and aligned for maximum correspondence over a comparison window or designated region as measured using a BLAST or BLAST 2.0 sequence comparison algorithms with default parameters described below, or by manual alignment and visual inspection (e.g., NCBI web site). Such sequences are then said to be “substantially identical.” This term also refers to, or can be applied to, the complement of a test sequence. The term also includes sequences that have deletions and/or additions, as well as those that have substitutions. In some embodiments, identity exists over a region that is at least about 25 amino acids or nucleotides in length, or 50-100 amino acids or nucleotides in length.

As used herein, the term “intact antibody” or “intact immunoglobulin” means an antibody that has at least two heavy (H) chain polypeptides and two light (L) chain polypeptides interconnected by disulfide bonds. Each heavy chain is comprised of a heavy chain variable region (abbreviated herein as HCVR or V_{H}) and a heavy chain constant region. The heavy chain constant region is comprised of three domains, CH₁, CH₂ and CH₃. Each light chain is comprised of a light chain variable region (abbreviated herein as LCVR or V_{L}) and a light chain constant region. The light chain constant region is comprised of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxyl-terminus in the following order: FR₁, CDR₁, FR₂, CDR₂, FR₃, CDR₃, FR₄. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (Clq) of the classical complement system.

As used herein, the terms “individual”, “patient”, or “subject” can be an individual organism, a vertebrate, a mammal, or a human. In some embodiments, the individual, patient or subject is a human.

The term “monoclonal antibody” as used herein refers to an antibody obtained from a population of substantially homogeneous antibodies, i.e., the individual antibodies comprising the population are identical except for possible naturally occurring mutations that may be present in minor amounts. For example, a monoclonal antibody can be an antibody that is derived from a single clone, including any eukaryotic, prokaryotic, or phage clone, and not the method by which it is produced. A monoclonal antibody composition displays a single binding specificity and affinity for a particular epitope. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. Monoclonal antibodies can be prepared using a wide variety of techniques known in the art including, e.g., but not limited to, hybridoma, recombinant, and phage display technologies. For example, the monoclonal antibodies to be used in accordance with the present methods may be made by the hybridoma method first described by Kohler et al., Nature 256:495 (1975), or may be made by recombinant DNA methods (See, e.g., U.S. Pat. No. 4,816,567). The “monoclonal antibodies” may also be isolated from phage antibody libraries using the techniques described in Clackson et al., Nature 352:624-628 (1991) and Marks et al., J. Mol. Biol. 222:581-597 (1991), for example.

As used herein, the term “pharmaceutically-acceptable carrier” is intended to include any and all solvents, dispersion media, coatings, antibacterial and antifungal compounds, isotonic and absorption delaying compounds, and the like, compatible with pharmaceutical administration. Pharmaceutically-acceptable carriers and their formulations are known to one skilled in the art and are described, for example, in Remington's Pharmaceutical Sciences (20^{th} edition, ed. A. Gennaro, 2000, Lippincott, Williams & Wilkins, Philadelphia, Pa.).

As used herein, “prevention” or “preventing” of a disorder or condition refers to a compound that, in a statistical sample, reduces the occurrence of the disorder or condition in the treated sample relative to an untreated control sample, or delays the onset or reduces the severity of one or more symptoms of the disorder or condition relative to the untreated control sample.

As used herein, the terms “polypeptide,” “peptide,” and “protein” are used interchangeably herein to mean a polymer comprising two or more amino acids joined to each other by peptide bonds or modified peptide bonds, i.e., peptide isosteres. Polypeptide refers to both short chains, commonly referred to as peptides, glycopeptides or oligomers, and to longer chains, generally referred to as proteins. Polypeptides may contain amino acids other than the 20 gene-encoded amino acids. Polypeptides include amino acid sequences modified either by natural processes, such as post-translational processing, or by chemical modification techniques that are well known in the art.

As used herein, the term “separate” therapeutic use refers to an administration of at least two active ingredients at the same time or at substantially the same time by different routes.

As used herein, the term “sequential” therapeutic use refers to administration of at least two active ingredients at different times, the administration route being identical or different. More particularly, sequential use refers to the whole administration of one of the active ingredients before administration of the other or others commences. It is thus possible to administer one of the active ingredients over several minutes, hours, or days before administering the other active ingredient or ingredients. There is no simultaneous treatment in this case.

As used herein, the term “simultaneous” therapeutic use refers to the administration of at least two active ingredients by the same route and at the same time or at substantially the same time.

As used herein, “specifically binds” refers to a molecule (e.g., an antibody or antigen binding fragment thereof) which recognizes and binds another molecule (e.g., an antigen), but that does not substantially recognize and bind other molecules. The terms “specific binding,” “specifically binds to,” or is “specific for” a particular molecule (e.g., a polypeptide, or an epitope on a polypeptide), as used herein, can be exhibited, for example, by a molecule having a K_{D} for the molecule to which it binds to of about 10⁻⁴ M, 10⁻⁵ M, 10⁻⁶ M, 10⁻⁷ M, 10⁻⁸ M, 10⁻⁹ M, 10⁻¹⁰ M, 10⁻¹¹ M, or 10⁻¹² M. The term “specifically binds” may also refer to binding where a molecule (e.g., an antibody or antigen binding fragment thereof) binds to a particular polypeptide (e.g., a ASIC1a polypeptide), or an epitope on a particular polypeptide, without substantially binding to any other polypeptide, or polypeptide epitope.

As used herein, the terms “subject,” “individual,” or “patient” can be an individual organism, a vertebrate, a mammal, or a human.

As used herein, the terms “treating” or “treatment” or “alleviation” refers to both therapeutic treatment and prophylactic or preventative measures, wherein the object is to prevent or slow down (lessen) the targeted pathologic condition or disorder. A subject is successfully “treated” for ischemic stroke, if, after receiving a therapeutic amount of the anti-ASIC1a antibodies of the present technology according to the methods described herein, the subject shows observable and/or measurable inhibition of the. It is also to be appreciated that the various modes of treatment or prevention of medical conditions as described are intended to mean “substantial”, which includes total but also less than total treatment or prevention, and wherein some biologically or medically relevant result is achieved.

It is also to be appreciated that the various modes of treatment of disorders as described herein are intended to mean “substantial,” which includes total but also less than total treatment, and wherein some biologically or medically relevant result is achieved. The treatment may be a continuous prolonged treatment for a chronic disease or a single, or few time administrations for the treatment of an acute condition.

Amino acid sequence modification(s) of the anti-ASIC1a antibodies described herein are contemplated. For example, it may be desirable to improve the binding affinity and/or other biological properties of the antibody. Amino acid sequence variants of an anti-ASIC1a antibody are prepared by introducing appropriate nucleotide changes into the antibody nucleic acid, or by peptide synthesis. Such modifications include, for example, deletions from, and/or insertions into and/or substitutions of, residues within the amino acid sequences of the antibody. Any combination of deletion, insertion, and substitution is made to obtain the antibody of interest, as long as the obtained antibody possesses the desired properties. The modification also includes the change of the pattern of glycosylation of the protein. The sites of greatest interest for substitutional mutagenesis include the hypervariable regions, but FR alterations are also contemplated. “Conservative substitutions” are shown in the Table below.

Amino Acid Substitutions

| Original | | Conservative |
|---|---|---|
| Residue | Exemplary Substitutions | Substitutions |
| Ala (A) | val; leu; ile | val |
| Arg (R) | lys; gln; asn | lys |
| Asn (N) | gln; his; asp, lys; arg | gln |
| Asp (D) | glu; asn | glu |
| Cys (C) | ser; ala | ser |
| Gln (Q) | asn; glu | asn |
| Glu (E) | asp; gln | asp |
| Gly (G) | ala | ala |
| His (H) | asn; gln; lys; arg | arg |
| Ile (I) | leu; val; met; ala; phe; norleucine | leu |
| Leu (L) | norleucine; ile; val; met; ala; phe | ile |
| Lys (K) | arg; gln; asn | arg |
| Met (M) | leu; phe; ile | leu |
| Phe (F) | leu; val; ile; ala; tyr | tyr |
| Pro (P) | ala | ala |
| Ser (S) | thr | thr |
| Thr (T) | ser | ser |
| Trp (W) | tyr; phe | tyr |
| Tyr (Y) | trp; phe; thr; ser | phe |
| Val (V) | ile; leu; met; phe; ala; norleucine | leu |

One type of substitutional variant involves substituting one or more hypervariable region residues of a parent antibody. A convenient way for generating such substitutional variants involves affinity maturation using phage display. Specifically, several hypervariable region sites (e.g., 6-7 sites) are mutated to generate all possible amino acid substitutions at each site. The antibody variants thus generated are displayed in a monovalent fashion from filamentous phage particles as fusions to the gene III product of M13 packaged within each particle. The phage-displayed variants are then screened for their biological activity (e.g., binding affinity) as herein disclosed. In order to identify candidate hypervariable region sites for modification, alanine scanning mutagenesis can be performed to identify hypervariable region residues contributing significantly to antigen binding. Alternatively, or additionally, it may be beneficial to analyze a crystal structure of the antigen-antibody complex to identify contact points between the antibody and the antigen. Such contact residues and neighboring residues are candidates for substitution according to the techniques elaborated herein. Once such variants are generated, the panel of variants is subjected to screening as described herein and antibodies with similar or superior properties in one or more relevant assays may be selected for further development.

Disease States with Altered ASIC1 Activity

Increasing evidence supports roles for ASICs in rodent models of pain, neurological disease and psychiatric disease. Wemmie et al., Nat Rev Neurosci. 14(7): 461-471 (2013). The activity of ASIC1a is controlled by ligands such as the neuropeptides (e.g., Dynorpin A and big dynorphin), polyamines (e.g., spermine), cations (e.g., Ca²⁺, Mg²⁺, Cd²⁺, Cu²⁺, Gd³⁺, Ni²⁺, Pb²⁺, Zn²⁺, Ba²⁺), toxins (PcT×1, MitTx and Mambalgin-1). Accumulating evidence suggests that acidosis potentiates cell death, and the diseases featuring altered ASIC1a activity include ischemic stroke, neurodegenerative disease, neuropsychological disease, epilepsy, multiple sclerosis, pain and migraine. Wemmie et al., Proc Natl Acad Sci USA. 101(10):3621-6 (2004); Coryell et al., J Neurosci. 29(17):5381-8 (2009); Xiong et al., Cell 118(6):687-98 (2004); Pignataro et al., Brain 130(Pt 1):151-8 (2007); Duan et al., J Neurosci. 31(6):2101-12 (2011); Friese et al., Nat Med. 13(12):1483-9 (2007); Vergo et al., Brain 134(Pt 2):571-84 (2011); Arun et al., Brain 136(Pt 1):106-15 (2013); and Duan et al., J Neurosci. 27(41):11139-48 (2007). Accordingly, antagonizing ASIC1 activity is a therapeutic approach for the treatment of these diseases. Interestingly, NSAIDS, such as flurbiprofen, ibuprofen, aspirin, salicylic acid, diclofenac, are known to reduce the ASIC1a currents. In some embodiments, altered ASIC1a activity is increased ASIC1a activity. Wemmie et al., Proc Natl Acad Sci USA. 101(10):3621-6 (2004); Duan et al., J Neurosci. 27(41):11139-48 (2007); Vergo et al., Brain 134(Pt 2):571-84 (2011); Duan et al., J Neurosci. 31(6):2101-12 (2011); and Arun et al., Brain 136(Pt 1):106-15 (2013).

### Pathogenesis of Ischemic Stroke

A stroke is caused by interruption or reduction of the supply of oxygen-rich blood to a part of the brain. Without oxygen, brain cells start to die within a few minutes. Stroke is usually presented through the symptoms such as sudden weakness; paralysis or numbness of the face, arms, or legs, especially on one side of the body; drooping of one side of the face; confusion; difficulty with speaking, such as slurred words, or difficulty understanding speech; trouble seeing in one or both eyes, such as blurred or blackened vision, or double vision in one or both eyes; problems with breathing; dizziness; difficulty with walking; loss of balance or coordination, causing, e.g., unexplained falls; loss of consciousness, and sudden and severe headache. Symptoms which are most common include sudden-onset face weakness (e.g., drooping of one side of the face), arm drift and abnormal speech. These symptoms typically start suddenly, over seconds to minutes, and in most cases do not progress further. Immediate emergency treatment is critical to surviving a stroke with the least amount of damage to the brain and ability to function.

Ischemic strokes, which account for about 87% of all strokes, occur when blood supply to a part of the brain is cut off. The decrease in cerebral blood flow (CBF), one of the primary responses of brain tissue to decrease in CBF is acidosis. A combination of hypoxia and glucose depletion leads to the decrease in ATP content in the ischemized brain area. Decrease in ATP leads to compensatory activation of anaerobic glycolysis and to increased production of lactate and H+ that causes the development of lactic acidosis. Modest increase in H+ concentration in early stages of ischemia plays a compensatory and adaptive role as it promotes the improvement of perfusion in the penumbral area. Significant elevation of lactate level within the first hours of ischemic stroke leads to decrease in extracellular pH can fall from 7.2 to below 6.5 in the core during focal cerebral ischemia. Acidosis appears to be an unfavorable prognostic sign for ischemic stroke.

Ischemic strokes occur when blood supply to a part of the brain is cut off because of obstruction of the blood vessels by blood clots or other particles. Fatty deposits called plaque can also cause blockages by building up in the blood vessels. The blocked blood flow in an ischemic stroke may be caused by atherosclerosis, which causes narrowing of the arteries over time. Ischemic strokes can be caused by a blockage anywhere along the arteries feeding the brain.

An ischemic stroke may be an embolic stroke, where a blood clot or plaque fragment forms somewhere else in the body (usually the heart) and travels to the brain. Once in the brain, the clot travels to a blood vessel small enough to block its passage. The clot lodges there, blocking the blood vessel and causing a stroke. About 15% of embolic strokes occur in people with atrial fibrillation (Afib).

An ischemic stroke may be a thrombotic stroke, which is caused by a blood clot that forms inside one of the arteries supplying blood to the brain. This type of stroke is usually seen in people with high cholesterol levels and atherosclerosis. Thrombotic stroke may be large vessel thrombosis or small vessel disease. Large vessel thrombosis occurs in the brain's larger arteries. In most cases it is caused by long-term atherosclerosis in combination with rapid blood clot formation. High cholesterol is a common risk factor for this type of stroke. Small vessel disease or lacunar infarction is closely linked to high blood pressure.

### Anti-ASICJa Antibodies of the Present Technology

The present technology describes methods and compositions for the generation and use of anti-ASIC1a antibodies of the present technology (e.g., anti-ASIC1a antibodies or antigen binding fragments thereof). The anti-ASIC1a antibodies of the present technology may be useful in the diagnosis, or treatment of ischemic stroke. Anti-ASIC1a antibodies of the present technology within the scope of the present technology include, e.g., but are not limited to, monoclonal, chimeric, humanized, bispecific antibodies and diabodies that specifically bind the target polypeptide, a homolog, derivative or a fragment thereof.

The Table below provides the complementarity determining region (CDR) sequences of the anti-ASIC1a antibodies of the present technology:

Accordingly, the antibody or antigen binding fragment thereof (anti-ASIC1a antibodies of the present technology) may comprise a heavy chain immunoglobulin variable domain (V_{H}) and a light chain immunoglobulin variable domain (V_{L}), wherein the V_{H} comprises complementarity determining regions V_{H}-CDR1, V_{H}—CDR2 and V_{H}-CDR3 disclosed herein; and wherein the V_{L} comprises complementarity determining regions V_{L}-CDR1, V_{L}—CDR2 and V_{L}—CDR3 disclosed herein.

The sequences of V_{H}-CDR1, V_{H}—CDR2, V_{H}—CDR3, V_{L}—CDR1, V_{L}—CDR2 and V_{L}-CDR3 of ASC06 are as follows:

CDR sequences of ASC06

| | | |
|---|---|---|
| SEQ ID NO: 3 | V_{L}-CDR1 | TGTSSDVGAYNYVSW |
| SEQ ID NO: 4 | V_{L}-CDR2 | GVSNRPS |
| SEQ ID NO: 5 | V_{L}-CDR3 | SSYTSSSTYV |
| SEQ ID NO: 8 | V_{H}-CDR1 | GFTFSSYAMS |
| SEQ ID NO: 9 | V_{H}-CDR2 | AISGSGGSTYYADSVKG |
| SEQ ID NO: 10 | V_{H}-CDR3 | DSFYGYSKGD |

The following sequences represent the V_{H}-CDR1, V_{H}—CDR2, V_{L}—CDR1, V_{L}—CDR2 and V_{L}—CDR3 sequences of ASC06-01 to ASC06-14:

Amino Acid Sequences of V_{H}-CDR1, V_{H}-CDR2,
V_{L}-CDR1, V_{L}-CDR2 and V_{L}-CDR3 of ASC06-01
to ASC06-14

| | | |
|---|---|---|
| SEQ ID NO: 3 | V_{L}-CDR1 | TGTSSDVGAYNYVSW |
| SEQ ID NO: 4 | V_{L}-CDR2 | GVSNRPS |
| SEQ ID NO: 5 | V_{L}-CDR3 | SSYTSSSTYV |
| SEQ ID NO: 8 | V_{L}-CDR1 | GFTFSSYAMS |
| SEQ ID NO: 9 | V_{H}-CDR2 | AISGSGGSTYYADSVKG |

In some embodiments, ASC06-01 to ASC06-14 comprise a V_{L} domain comprising an amino acid sequence set forth in SEQ ID NO: 2. The V_{H}-CDR3 sequences ASC06-01 to ASC06-14 are as shown in the Table below.

Amino Acid Sequences of V_{H}-CDR3 of
ASC06-01 to ASC06-14

| Name | Amino Acid Sequence of V_{H}-CDR3 |
|---|---|
| ASC06-01 | DSYFGYSKGD (SEQ ID NO: 11) |
| ASC06-02 | DSFFGRAKGS (SEQ ID NO: 13) |
| ASC06-03 | DSFYGRAKGS (SEQ ID NO: 15) |
| ASC06-04 | DSFYGRAKGV (SEQ ID NO: 17) |
| ASC06-05 | DSYFGRAKGS (SEQ ID NO: 19) |
| ASC06-06 | DSFYGRAKGD (SEQ ID NO: 21) |
| ASC06-07 | DSFYGYAKGL (SEQ ID NO: 23) |
| ASC06-08 | DSFFGWAKGV (SEQ ID NO: 25) |
| ASC06-09 | DSFYGRSKGI (SEQ ID NO: 27) |
| ASC06-10 | DSFYGWAKGL (SEQ ID NO: 29) |
| ASCO6-11 | DSFYGRAKGK (SEQ ID NO: 31) |
| ASCO6-12 | DSFFGRAKGL (SEQ ID NO: 33) |
| ASC06-13 | VSFFGWAKGD (SEQ ID NO: 35) |
| ASC06-14 | DSFFGYAKGH (SEQ ID NO: 37) |

In one aspect, the present technology relates to an antibody, or antigen binding fragment thereof comprising a heavy chain immunoglobulin variable domain (V_{H}) and a light chain immunoglobulin variable domain (V_{L}), wherein the V_{H} comprises a V_{H}-CDR1 sequence of SEQ ID NO: 8, a V_{H}—CDR2 sequence of SEQ ID NO: 9, and a V_{H}-CDR3 sequence selected from the group consisting of: SEQ ID NO: 10, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, and SEQ ID NO: 37; and wherein the V_{L} comprises a V_{L}-CDR1 sequence of SEQ ID NO: 3, a V_{L}—CDR2 sequence of SEQ ID NO: 4, and a V_{L}-CDR3 sequence of SEQ ID NO: 5.

Additionally, or alternatively, in some embodiments, the antibody, or antigen binding fragment thereof further comprises a Fc domain of an isotype selected from the group consisting of IgG1, IgG2, IgG3, IgG4, IgA1, IgA2, IgM, IgD, and IgE. Additionally, or alternatively, in some embodiments, the antigen binding fragment is selected from the group consisting of Fab, F(ab′)2, Fab′, scFv, and Fv. Additionally, or alternatively, in some embodiments, the antibody is a monoclonal antibody, a chimeric antibody, a humanized antibody, or a bispecific antibody.

Additionally, or alternatively, in some embodiments, the antibody, or antigen binding fragment thereof binds to ASIC1a. Additionally, or alternatively, in some embodiments, the antibody, or antigen binding fragment thereof is an antagonist of ASIC1a. Additionally, or alternatively, in some embodiments, the antibody, or antigen binding fragment thereof inhibits ASIC1a-mediated, acid-induced currents. Additionally, or alternatively, in some embodiments, the antibody, or antigen binding fragment thereof inhibits ASIC1a-mediated, acid-induced calcium influx.

Additionally, or alternatively the VL comprises SEQ ID NO: 2; and the V_{H} Comprises: a V_{H}-CDR1 sequence of SEQ ID NO: 8, a V_{H}—CDR2 sequence of SEQ ID NO: 9, and a V_{H}-CDR3 sequence of SEQ ID NO: 11; a V_{H}-CDR1 sequence of SEQ ID NO: 8, a V_{H}—CDR2 sequence of SEQ ID NO: 9, and a V_{H}-CDR3 sequence of SEQ ID NO: 13; a V_{H}-CDR1 sequence of SEQ ID NO: 8, a V_{H}—CDR2 sequence of SEQ ID NO: 9, and a V_{H}-CDR3 sequence of SEQ ID NO: 15; a V_{H}-CDR1 sequence of SEQ ID NO: 8, a V_{H}—CDR2 sequence of SEQ ID NO: 9, and a V_{H}-CDR3 sequence of SEQ ID NO: 17; a V_{H}-CDR1 sequence of SEQ ID NO: 8, a V_{H}—CDR2 sequence of SEQ ID NO: 9, and a V_{H}-CDR3 sequence of SEQ ID NO: 19; a V_{H}-CDR1 sequence of SEQ ID NO: 8, a V_{H}—CDR2 sequence of SEQ ID NO: 9, and a V_{H}-CDR3 sequence of SEQ ID NO: 21; a V_{H}-CDR1 sequence of SEQ ID NO: 8, a V_{H}-CDR2 sequence of SEQ ID NO: 9, and a V_{H}-CDR3 sequence of SEQ ID NO: 23; a V_{H}-CDR1 sequence of SEQ ID NO: 8, a V_{H}—CDR2 sequence of SEQ ID NO: 9, and a V_{H}-CDR3 sequence of SEQ ID NO: 25; a V_{H}-CDR1 sequence of SEQ ID NO: 8, a V_{H}—CDR2 sequence of SEQ ID NO: 9, and a V_{H}-CDR3 sequence of SEQ ID NO: 27; a V_{H}-CDR1 sequence of SEQ ID NO: 8, a V_{H}—CDR2 sequence of SEQ ID NO: 9, and a V_{H}-CDR3 sequence of SEQ ID NO:29; a V_{H}-CDR1 sequence of SEQ ID NO: 8, a V_{H}—CDR2 sequence of SEQ ID NO: 9, and a V_{H}-CDR3 sequence of SEQ ID NO: 31; a V_{H}-CDR1 sequence of SEQ ID NO: 8, a V_{H}-CDR2 sequence of SEQ ID NO: 9, and a V_{H}-CDR3 sequence of SEQ ID NO: 33; a V_{H}-CDR1 sequence of SEQ ID NO: 8, a V_{H}—CDR2 sequence of SEQ ID NO: 9, and a V_{H}-CDR3 sequence of SEQ ID NO: 35; or a V_{H}—CDR1 sequence of SEQ ID NO: 8, a V_{H}—CDR2 sequence of SEQ ID NO: 9, and a V_{H}-CDR3 sequence of SEQ ID NO: 37.

Additionally, or alternatively, in some embodiments, the V_{L} comprises a V_{L}-CDR1 sequence of SEQ ID NO: 3, a V_{L}—CDR2 sequence of SEQ ID NO: 4, a V_{L}—CDR3 sequence of SEQ ID NO: 5, and the V_{H} comprises: a V_{H}-CDR1 sequence of SEQ ID NO: 8, a V_{H}—CDR2 sequence of SEQ ID NO: 9, and a V_{H}-CDR3 sequence of SEQ ID NO: 11; a V_{H}-CDR1 sequence of SEQ ID NO: 8, a V_{H}—CDR2 sequence of SEQ ID NO: 9, and a V_{H}-CDR3 sequence of SEQ ID NO: 13; a V_{H}-CDR1 sequence of SEQ ID NO: 8, a V_{H}—CDR2 sequence of SEQ ID NO: 9, and a V_{H}-CDR3 sequence of SEQ ID NO: 15; a V_{H}-CDR1 sequence of SEQ ID NO: 8, a V_{H}—CDR2 sequence of SEQ ID NO: 9, and a V_{H}-CDR3 sequence of SEQ ID NO: 17; a V_{H}-CDR1 sequence of SEQ ID NO: 8, a V_{H}—CDR2 sequence of SEQ ID NO: 9, and a V_{H}-CDR3 sequence of SEQ ID NO: 19; a V_{H}-CDR1 sequence of SEQ ID NO: 8, a V_{H}-CDR2 sequence of SEQ ID NO: 9, and a V_{H}-CDR3 sequence of SEQ ID NO: 21; a V_{H}-CDR1 sequence of SEQ ID NO: 8, a V_{H}—CDR2 sequence of SEQ ID NO: 9, and a V_{H}-CDR3 sequence of SEQ ID NO: 23; a V_{H}-CDR1 sequence of SEQ ID NO: 8, a V_{H}—CDR2 sequence of SEQ ID NO: 9, and a V_{H}-CDR3 sequence of SEQ ID NO: 25; a V_{H}-CDR1 sequence of SEQ ID NO: 8, a V_{H}—CDR2 sequence of SEQ ID NO: 9, and a V_{H}-CDR3 sequence of SEQ ID NO:27; a V_{H}-CDR1 sequence of SEQ ID NO: 8, a V_{H}—CDR2 sequence of SEQ ID NO: 9, and a V_{H}-CDR3 sequence of SEQ ID NO:29; a V_{H}-CDR1 sequence of SEQ ID NO: 8, a V_{H}-CDR2 sequence of SEQ ID NO: 9, and a V_{H}-CDR3 sequence of SEQ ID NO: 31; a V_{H}-CDR1 sequence of SEQ ID NO: 8, a V_{H}—CDR2 sequence of SEQ ID NO: 9, and a V_{H}-CDR3 sequence of SEQ ID NO:33; a V_{H}-CDR1 sequence of SEQ ID NO: 8, a V_{H}—CDR2 sequence of SEQ ID NO: 9, and a V_{H}-CDR3 sequence of SEQ ID NO: 35; or a V_{H}—CDR1 sequence of SEQ ID NO: 8, a V_{H}—CDR2 sequence of SEQ ID NO: 9, and a V_{H}-CDR3 sequence of SEQ ID NO: 37.

In one aspect, the present technology relates to an antibody, or antigen binding fragment thereof comprising a heavy chain immunoglobulin variable domain (V_{H}) and a light chain immunoglobulin variable domain (V_{L}), wherein the V_{H} Comprises an amino acid sequence of SEQ ID NO:7 and the V_{L} comprises an amino acid sequence of SEQ ID NO: 2. In one aspect, the present disclosure provides an antibody, or antigen binding fragment thereof comprising (a) a light chain immunoglobulin variable domain sequence (V_{L}) that is at least 80%, at least 85%, at least 90%, at least 95%, or at least 99% identical to the light chain immunoglobulin variable domain sequence of SEQ ID NO: 2; and/or (b) a heavy chain immunoglobulin variable domain sequence (V_{H}) that is at least 80%, at least 85%, at least 90%, at least 95%, or at least 99% identical to the heavy chain immunoglobulin variable domain sequence of SEQ ID NO: 7.

In one aspect, the present technology relates to an antibody, or antigen binding fragment thereof comprising antibody, or antigen binding fragment thereof, comprising a light chain (LC) and a heavy chain (HC), wherein the LC comprises an amino acid sequence comprising SEQ ID NO: 2, and wherein HC comprises a heavy chain immunoglobulin variable domain (V_{H}), wherein the V_{H} comprises a V_{H}-CDR1 sequence of SEQ ID NO: 8, a V_{H}—CDR2 sequence of SEQ ID NO: 9, and a V_{H}-CDR3 sequence selected from the group consisting of: SEQ ID NO: 10, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, and SEQ ID NO: 37.

In one aspect, the present disclosure provides an antibody, or antigen binding fragment thereof comprising (a) a light chain immunoglobulin variable domain sequence (V_{L}) that is at least 80%, at least 85%, at least 90%, at least 95%, or at least 99% identical to the light chain immunoglobulin variable domain sequence present in SEQ ID NO: 2; and/or (b) a heavy chain immunoglobulin variable domain sequence (V_{H}) comprising (b1) a V_{H}-CDR1 that is at least 80%, at least 85%, at least 90%, at least 95%, or at least 99% identical to the V_{H}-CDR1 present in SEQ ID NO: 8; (b2) a V_{H}-CDR2 that is at least 80%, at least 85%, at least 90%, at least 95%, or at least 99% identical to the V_{H}-CDR2 present in SEQ ID NO: 9, and/or (b3) a V_{H}-CDR3 that is at least 80%, at least 85%, at least 90%, at least 95%, or at least 99% identical to the V_{H}-CDR3 present in any one of SEQ ID NO: 10, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, or SEQ ID NO: 37.

Additionally, or alternatively, in some embodiments, theV_{L} comprises a V_{L}-CDR1 that is at least 80%, at least 85%, at least 90%, at least 95%, or at least 99% identical to the V_{H}-CDR1 present in SEQ ID NO: 3; a V_{L}-CDR2 that is at least 80%, at least 85%, at least 90%, at least 95%, or at least 99% identical to the V_{H}-CDR2 present in SEQ ID NO: 4; and/or a V_{L}—CDR3 that is at least 80%, at least 85%, at least 90%, at least 95%, or at least 99% identical to the V_{H}-CDR3 present in SEQ ID NO: 5.

In one aspect, the present technology relates to an antibody, or antigen binding fragment thereof comprising a heavy chain immunoglobulin variable domain (V_{H}) and a light chain immunoglobulin variable domain (V_{L}), wherein the V_{L} Comprises an amino acid sequence of SEQ ID NO: 2. Additionally, or alternatively, in some embodiments, the V_{H} comprises: a V_{H}-CDR1 sequence of SEQ ID NO: 8, a V_{H}—CDR2 sequence of SEQ ID NO: 9, and a V_{H}-CDR3 sequence of SEQ ID NO: 11; a V_{H}-CDR1 sequence of SEQ ID NO: 8, a V_{H}-CDR2 sequence of SEQ ID NO: 9, and a V_{H}-CDR3 sequence of SEQ ID NO: 13; a V_{H}-CDR1 sequence of SEQ ID NO: 8, a V_{H}—CDR2 sequence of SEQ ID NO: 9, and a V_{H}-CDR3 sequence of SEQ ID NO: 15; a V_{H}-CDR1 sequence of SEQ ID NO: 8, a V_{H}—CDR2 sequence of SEQ ID NO: 9, and a V_{H}-CDR3 sequence of SEQ ID NO: 17; a V_{H}-CDR1 sequence of SEQ ID NO: 8, a V_{H}—CDR2 sequence of SEQ ID NO: 9, and a V_{H}-CDR3 sequence of SEQ ID NO: 19; a V_{H}-CDR1 sequence of SEQ ID NO: 8, a V_{H}—CDR2 sequence of SEQ ID NO: 9, and a V_{H}-CDR3 sequence of SEQ ID NO: 21; a V_{H}-CDR1 sequence of SEQ ID NO: 8, a V_{H}-CDR2 sequence of SEQ ID NO: 9, and a V_{H}-CDR3 sequence of SEQ ID NO: 23; a V_{H}-CDR1 sequence of SEQ ID NO: 8, a V_{H}—CDR2 sequence of SEQ ID NO: 9, and a V_{H}-CDR3 sequence of SEQ ID NO: 25; a V_{H}-CDR1 sequence of SEQ ID NO: 8, a V_{H}—CDR2 sequence of SEQ ID NO: 9, and a V_{H}-CDR3 sequence of SEQ ID NO:27; a V_{H}-CDR1 sequence of SEQ ID NO: 8, a V_{H}—CDR2 sequence of SEQ ID NO: 9, and a V_{H}-CDR3 sequence of SEQ ID NO:29; a V_{H}-CDR1 sequence of SEQ ID NO: 8, a V_{H}—CDR2 sequence of SEQ ID NO: 9, and a V_{H}-CDR3 sequence of SEQ ID NO: 31; a V_{H}-CDR1 sequence of SEQ ID NO: 8, a V_{H}-CDR2 sequence of SEQ ID NO: 9, and a V_{H}-CDR3 sequence of SEQ ID NO:33; a V_{H}-CDR1 sequence of SEQ ID NO: 8, a V_{H}—CDR2 sequence of SEQ ID NO: 9, and a V_{H}-CDR3 sequence of SEQ ID NO: 35; or a V_{H}—CDR1 sequence of SEQ ID NO: 8, a V_{H}—CDR2 sequence of SEQ ID NO: 9, and a V_{H}-CDR3 sequence of SEQ ID NO: 37.

Additionally, or alternatively, in some embodiments, the antibody further comprises an amino acid sequence selected from the group consisting of: SEQ ID NO: 8 and SEQ ID NO: 9. Additionally, or alternatively, in some embodiments, the antibody comprises the amino acid sequences of SEQ ID NO: 8 and SEQ ID NO: 9.

Additionally, or alternatively, in some embodiments, the antibody further comprises a Fe domain of an isotype selected from the group consisting of IgG1, IgG2, IgG3, IgG4, IgA1, IgA2, IgM, IgD, and IgE. Additionally, or alternatively, in some embodiments, the antibody is a monoclonal antibody, a chimeric antibody, a humanized antibody, or a bispecific antibody.

Additionally, or alternatively, in some embodiments, the antibody binds to ASIC1a. Additionally, or alternatively, in some embodiments, the antibody is an antagonist of ASIC1a. Additionally, or alternatively, in some embodiments, the antibody inhibits ASIC1a-mediated, acid-induced currents. Additionally, or alternatively, in some embodiments, the antibody inhibits ASIC1a-mediated, acid-induced calcium influx.

In one aspect, the present technology provides a nucleic acid sequence encoding any of the immunoglobulin-related compositions described herein. Also disclosed herein are recombinant nucleic acid sequences encoding any of the antibodies described herein. In some embodiments, the nucleic acid sequence is selected from the group consisting of SEQ ID NOs: 1, 6, 12, 14, 16, 18, 20, 22, 24, 26, 28, 30, 32, 34, 36 and 38.

In another aspect, the present technology provides a host cell expressing any nucleic acid sequence encoding any of the immunoglobulin-related compositions described herein.

The antibody or antigen binding fragment thereof (anti-ASIC1a antibodies of the present technology) may specifically bind ASIC1a protein. In some embodiments, the anti-ASIC1a antibodies of the present technology may bind the extracellular domain of the ASIC1a. In some embodiments, the anti-ASIC1a antibodies of the present technology may bind an epitope that spans two ASIC1a monomers.

In some embodiments, the anti-ASIC1a antibodies of the present technology inhibit the function of ASIC1a trimer. In some embodiments, the anti-ASIC1a antibodies of the present technology decrease the stability of ASIC1a trimer. In some embodiments, the anti-ASIC1a antibodies of the present technology enhance the function of ASIC1a trimer. In some embodiments, the anti-ASIC1a antibodies of the present technology stabilize ASIC1a trimer. In some embodiments, the anti-ASIC1a antibodies of the present technology inhibit heterooligomerization (e.g. heterotrimerization) of ASIC1a with other ASIC1 isomers.

In some embodiments, the antibody or antigen binding fragment thereof is an antibody, scFv, (scFv)₂, Fab, Fab′, F(ab′)₂ or an scFv-Fc antibody. In some embodiments, the antibody or antigen binding fragment thereof is an scFv antibody. In some embodiments, the scFv antibody is ASC06, ASC06-01 ASC06-02 ASC06-03 ASC06-04 ASC06-05 ASC06-06 ASC06-07 ASC06-08 ASC06-09 ASC06-10 ASC06-11 ASC06-12 ASC06-13 or ASC06-14.

### Formulations

By way of an example, anti-ASIC1a antibodies of the present technology is formulated in a simple delivery vehicle. However, anti-ASIC1a antibodies of the present technology may be lyophilized or incorporated in a gel, cream, biomaterial, sustained release delivery vehicle.

Anti-ASIC1a antibodies of the present technology are generally combined with a pharmaceutically acceptable carrier. The term “pharmaceutically acceptable carrier” refers to any pharmaceutical carrier that does not itself induce the production of antibodies harmful to the individual receiving the composition, and which can be administered without undue toxicity. Suitable carriers can be large, slowly metabolized macromolecules such as proteins, polysaccharides, polylactic acids, polyglycolic acids, polymeric amino acids and amino acid copolymers. Such carriers are well known to those of ordinary skill in the art. Pharmaceutically acceptable carriers in therapeutic compositions can include liquids such as water, saline, glycerol and ethanol. Auxiliary substances, such as wetting or emulsifying agents, pH buffering substances, and the like, can also be present in such vehicles. Pharmaceutically acceptable salts can also be present in the pharmaceutical composition, e.g. mineral acid salts such as hydrochlorides, hydrobromides, phosphates, sulfates, and the like; and the salts of organic acids such as acetates, propionates, malonates, benzoates, and the like.

### Modes of Administration and Effective Dosages

Any method known to those in the art for contacting a cell, organ or tissue with a peptide may be employed. Suitable methods include in vitro, ex vivo, or in vivo methods. In vivo methods typically include the administration of an anti-ASIC1a antibodies of the present technology, such as those described above, to a mammal, suitably a human. When used in vivo for therapy, the anti-ASIC1a antibodies of the present technology are administered to the subject in effective amounts (i.e., amounts that have desired therapeutic effect). The dose and dosage regimen will depend upon the degree of the infection in the subject, the characteristics of the particular anti-ASIC1a antibodies of the present technology used, e.g., its therapeutic index, the subject, and the subject's history.

The effective amount may be determined during pre-clinical trials and clinical trials by methods familiar to physicians and clinicians. An effective amount of a peptide useful in the methods may be administered to a mammal in need thereof by any of a number of well-known methods for administering pharmaceutical compounds. The peptide may be administered systemically or locally.

The anti-ASIC1a antibodies of the present technology described herein can be incorporated into pharmaceutical compositions for administration, singly or in combination, to a subject for the treatment or prevention of a disorder described herein. Such compositions typically include the active agent and a pharmaceutically acceptable carrier. As used herein the term “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can also be incorporated into the compositions.

Pharmaceutical compositions are typically formulated to be compatible with its intended route of administration. Examples of routes of administration include parenteral (e.g., intravenous, intradermal, intraperitoneal or subcutaneous), oral, inhalation, transdermal (topical), intraocular, iontophoretic, and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic. For convenience of the patient or treating physician, the dosing formulation can be provided in a kit containing all necessary equipment (e.g., vials of drug, vials of diluent, syringes and needles) for a treatment course (e.g., 7 days of treatment).

In some embodiments, the anti-ASIC1a antibodies of the present technology is administered by a parenteral route. In some embodiments, the antibody or antigen binding fragment thereof is administered by a topical route.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, a composition for parenteral administration must be sterile and should be fluid to the extent that easy syringability exists. It should be stable under the conditions of manufacture and storage and must be preserved against the contaminating action of microorganisms such as bacteria and fungi.

The anti-ASIC1a antibodies of the present technology compositions can include a carrier, which can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thiomerasol, and the like. Glutathione and other antioxidants can be included to prevent oxidation. In many cases, isotonic agents are included, for example, sugars, polyalcohols such as mannitol, sorbitol, or sodium chloride in the composition. Prolonged absorption of the injectable compositions can be brought about by including in the composition an agent which delays absorption, for example, aluminum monostearate or gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, typical methods of preparation include vacuum drying and freeze drying, which can yield a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash.

Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the anti-ASIC1a antibodies of the present technology can be delivered in the form of an aerosol spray from a pressurized container or dispenser which contains a suitable propellant, e.g., a gas such as carbon dioxide, or a nebulizer. Such methods include those described in U.S. Pat. No. 6,468,798.

Systemic administration of an anti-ASIC1a antibodies of the present technology as described herein can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays. For transdermal administration, the active compounds are formulated into ointments, salves, gels, or creams as generally known in the art. In one embodiment, transdermal administration may be performed by iontophoresis.

An anti-ASIC1a antibodies of the present technology can be formulated in a carrier system. The carrier can be a colloidal system. The colloidal system can be a liposome, a phospholipid bilayer vehicle. In one embodiment, the therapeutic peptide is encapsulated in a liposome while maintaining peptide integrity. As one skilled in the art would appreciate, there are a variety of methods to prepare liposomes. (See Lichtenberg et al., Methods Biochem. Anal., 33:337-462 (1988); Anselem et al., Liposome Technology, CRC Press (1993)). Liposomal formulations can delay clearance and increase cellular uptake (See Reddy, Ann. Pharmacother., 34(7-8):915-923 (2000)). An active agent can also be loaded into a particle prepared from pharmaceutically acceptable ingredients including, but not limited to, soluble, insoluble, permeable, impermeable, biodegradable or gastroretentive polymers or liposomes. Such particles include, but are not limited to, nanoparticles, biodegradable nanoparticles, microparticles, biodegradable microparticles, nanospheres, biodegradable nanospheres, microspheres, biodegradable microspheres, capsules, emulsions, liposomes, micelles and viral vector systems.

The carrier can also be a polymer, e.g., a biodegradable, biocompatible polymer matrix. In one embodiment, the anti-ASIC1a antibodies of the present technology can be embedded in the polymer matrix, while maintaining protein integrity. The polymer may be natural, such as polypeptides, proteins or polysaccharides, or synthetic, such as poly α-hydroxy acids. Examples include carriers made of, e.g., collagen, fibronectin, elastin, cellulose acetate, cellulose nitrate, polysaccharide, fibrin, gelatin, and combinations thereof.

In one embodiment, the polymer is poly-lactic acid (PLA) or copoly lactic/glycolic acid (PGLA). The polymeric matrices can be prepared and isolated in a variety of forms and sizes, including microspheres and nanospheres. Polymer formulations can lead to prolonged duration of therapeutic effect. (See Reddy, Ann. Pharmacother., 34(7-8):915-923 (2000)). A polymer formulation for human growth hormone (hGH) has been used in clinical trials. (See Kozarich and Rich, Chemical Biology, 2:548-552 (1998)).

Examples of polymer microsphere sustained release formulations are described in PCT publication WO 99/15154 (Tracy et al.), U.S. Pat. Nos. 5,674,534 and 5,716,644 (both to Zale et al.), PCT publication WO 96/40073 (Zale et al.), and PCT publication WO 00/38651 (Shah et al.). U.S. Pat. Nos. 5,674,534 and 5,716,644 and PCT publication WO 96/40073 describe a polymeric matrix containing particles of erythropoietin that are stabilized against aggregation with a salt.

In some embodiments, the anti-ASIC1a antibodies of the present technology are prepared with carriers that will protect the anti-ASIC1a antibodies of the present technology against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using known techniques. The materials can also be obtained commercially, e.g., from Alza Corporation and Nova Pharmaceuticals, Inc. Liposomal suspensions (including liposomes targeted to specific cells with monoclonal antibodies to cell-specific antigens) can also be used as pharmaceutically acceptable carriers. These can be prepared according to methods known to those skilled in the art, for example, as described in U.S. Pat. No. 4,522,811.

The anti-ASIC1a antibodies of the present technology can also be formulated to enhance intracellular delivery. For example, liposomal delivery systems are known in the art, see, e.g., Chonn and Cullis, “Recent Advances in Liposome Drug Delivery Systems,” Current Opinion in Biotechnology 6:698-708 (1995); Weiner, “Liposomes for Protein Delivery: Selecting Manufacture and Development Processes,” Immunomethods, 4(3):201-9 (1994); and Gregoriadis, “Engineering Liposomes for Drug Delivery: Progress and Problems,” Trends Biotechnol., 13(12):527-37 (1995). Mizguchi et al., Cancer Lett., 100:63-69 (1996), describes the use of fusogenic liposomes to deliver a protein to cells both in vivo and in vitro.

Dosage, toxicity and therapeutic efficacy of the anti-ASIC1a antibodies of the present technology can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. In some embodiments, the anti-ASIC1a antibodies of the present technology exhibit high therapeutic indices. While anti-ASIC1a antibodies of the present technology that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies within a range of circulating concentrations that include the ED50 with little or no toxicity. The dosage may vary within this range depending upon the dosage form employed and the route of administration utilized. For any anti-ASIC1a antibodies of the present technology used in the methods, the therapeutically effective dose can be estimated initially from cell culture assays. A dose can be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

Typically, an effective amount of the anti-ASIC1a antibodies of the present technology, sufficient for achieving a therapeutic or prophylactic effect, range from about 0.000001 mg per kilogram body weight per day to about 10,000 mg per kilogram body weight per day. Suitably, the dosage ranges are from about 0.0001 mg per kilogram body weight per day to about 100 mg per kilogram body weight per day. For example, dosages can be 1 mg/kg body weight or 10 mg/kg body weight every day, every two days or every three days or within the range of 1-10 mg/kg every week, every two weeks or every three weeks. In one embodiment, a single dosage of peptide ranges from 0.001-10,000 micrograms per kg body weight. In one embodiment, anti-ASIC1a antibodies of the present technology concentrations in a carrier range from 0.2 to 2000 micrograms per delivered milliliter. An exemplary treatment regime entails administration once per day or once a week. In therapeutic applications, a relatively high dosage at relatively short intervals is sometimes required until progression of the disease is reduced or terminated, and until the subject shows partial or complete amelioration of symptoms of disease. Thereafter, the patient can be administered a prophylactic regime.

In some embodiments, a therapeutically effective amount of an anti-ASIC1a antibodies of the present technology may be defined as a concentration of peptide at the target tissue of 10⁻¹² to 10⁻⁶ molar, e.g., approximately 10⁻⁷ molar. This concentration may be delivered by systemic doses of 0.001 to 100 mg/kg or equivalent dose by body surface area. The schedule of doses would be optimized to maintain the therapeutic concentration at the target tissue. In some embodiments, the doses are administered by single daily or weekly administration, but may also include continuous administration (e.g., parenteral infusion or transdermal application). In some embodiments, the dosage of the anti-ASIC1a antibodies of the present technology is provided at a “low,” “mid,” or “high” dose level. In one embodiment, the low dose is provided from about 0.0001 to about 0.5 mg/kg/h, suitably from about 0.001 to about 0.1 mg/kg/h. In one embodiment, the mid-dose is provided from about 0.01 to about 1.0 mg/kg/h, suitably from about 0.01 to about 0.5 mg/kg/h. In one embodiment, the high dose is provided from about 0.5 to about 10 mg/kg/h, suitably from about 0.5 to about 2 mg/kg/h.

For example, a therapeutically effective amount may partially or completely alleviate one or more symptoms of ischemic stroke, including sudden weakness; paralysis or numbness of the face, arms, or legs, especially on one side of the body; drooping of one side of the face; confusion; difficulty with speaking, such as slurred words, or difficulty understanding speech; trouble seeing in one or both eyes, such as blurred or blackened vision, or double vision in one or both eyes; problems with breathing; dizziness; difficulty with walking; loss of balance or coordination, causing, e.g., unexplained falls; loss of consciousness, and sudden and severe headache. A therapeutically effective amount may partially or completely alleviate one or more symptoms of ischemic stroke, including, but not limited to, sudden-onset face weakness (such as drooping of one side) of the face, arm drift and abnormal speech.

The skilled artisan will appreciate that certain factors may influence the dosage and timing required to effectively treat a subject, including but not limited to, the severity of the disease or disorder, previous treatments, the general health and/or age of the subject, and other diseases present. Moreover, treatment of a subject with a therapeutically effective amount of the therapeutic compositions described herein can include a single treatment or a series of treatments.

The mammal treated in accordance present methods can be any mammal, including, for example, farm animals, such as sheep, pigs, cows, and horses; pet animals, such as dogs and cats; laboratory animals, such as rats, mice and rabbits. In some embodiments, the mammal is a human.

### Use of the Anti-ASIC1a Antibodies of the Present Technology

General. The anti-ASIC1a antibodies of the present technology are useful in methods known in the art relating to the localization and/or quantitation of ASIC1a protein or a mutant thereof (e.g., for use in measuring levels of the ASIC1a protein within appropriate physiological samples, for use in diagnostic methods, for use in imaging the polypeptide, and the like). The anti-ASIC1a antibodies of the present technology are useful to isolate a ASIC1a protein by standard techniques, such as affinity chromatography or immunoprecipitation. The anti-ASIC1a antibodies of the present technology can facilitate the purification of natural immunoreactive ASIC1a protein from biological samples, e.g., mammalian sera or cells as well as recombinantly-produced immunoreactive ASIC1a protein expressed in a host system. Moreover, anti-ASIC1a antibodies of the present technology can be used to detect an immunoreactive ASIC1a protein or a fragment thereof (e.g., in plasma, a cellular lysate or cell supernatant) in order to evaluate the abundance and pattern of expression of the immunoreactive polypeptide. The anti-ASIC1a antibodies of the present technology can be used diagnostically to monitor immunoreactive ASIC1a protein levels in tissue as part of a clinical testing procedure, e.g., to determine the efficacy of a given treatment regimen. As noted above, the detection can be facilitated by coupling (i.e., physically linking) the anti-ASIC1a antibodies of the present technology to a detectable substance.

Detection of ASIC1a protein. An exemplary method for detecting the presence or absence of an immunoreactive ASIC1a protein in a biological sample involves obtaining a biological sample from a test subject and contacting the biological sample with the anti-ASIC1a antibodies of the present technology capable of detecting an immunoreactive ASIC1a protein such that the presence of an immunoreactive ASIC1a protein is detected in the biological sample. Detection may be accomplished by means of a detectable label attached to the antibody.

The term “labeled” with regard to the anti-ASIC1a antibodies of the present technology antibody is intended to encompass direct labeling of the antibody by coupling (i.e., physically linking) a detectable substance to the antibody, as well as indirect labeling of the antibody by reactivity with another compound that is directly labeled, such as a secondary antibody. Examples of indirect labeling include detection of a primary antibody using a fluorescently-labeled secondary antibody and end-labeling of a DNA probe with biotin such that it can be detected with fluorescently-labeled streptavidin.

In some embodiments, the anti-ASIC1a antibodies of the present technology disclosed herein are conjugated to one or more detectable labels. For such uses, the anti-ASIC1a antibodies of the present technology antibodies may be detectably labeled by covalent or non-covalent attachment of a chromogenic, enzymatic, radioisotopic, isotopic, fluorescent, toxic, chemiluminescent, nuclear magnetic resonance contrast agent or other label.

Examples of suitable chromogenic labels include diaminobenzidine and 4-hydroxyazo-benzene-2-carboxylic acid. Examples of suitable enzyme labels include malate dehydrogenase, staphylococcal nuclease, A-5-steroid isomerase, yeast-alcohol dehydrogenase, α-glycerol phosphate dehydrogenase, triose phosphate isomerase, peroxidase, alkaline phosphatase, asparaginase, glucose oxidase, O-galactosidase, ribonuclease, urease, catalase, glucose-6-phosphate dehydrogenase, glucoamylase, and acetylcholine esterase.

Examples of suitable radioisotopic labels include ³H, ¹¹¹In, ¹²⁵I, ¹³¹I, ³²P, ³⁵S, ¹⁴C, ⁵¹Cr, ⁵⁷To, ⁵⁸Co, ⁵⁹Fe, ⁷⁵Se, ¹⁵²Eu, ⁹⁰Y, ⁶⁷Cu, ²¹⁷Ci, ²¹¹At, ²¹²Pb, ⁴⁷Sc, ¹⁰⁹Pd, etc. ¹¹¹In is an exemplary isotope where in vivo imaging is used since its avoids the problem of dehalogenation of the ¹²⁵I or ¹³¹I-labeled ASIC1a-, or ASIC1a-protein binding antibodies by the liver. In addition, this isotope has a more favorable gamma emission energy for imaging (Perkins et al, Eur. J. Nucl. Med. 70:296-301 (1985); Carasquillo et al., J. Nucl. Med. 25:281-287 (1987)). For example, ¹¹¹In coupled to monoclonal antibodies with 1-(P-isothiocyanatobenzyl)-DPTA exhibits little uptake in non-tumorous tissues, particularly the liver, and enhances specificity of tumor localization (Esteban et al., J. Nucl. Med. 28:861-870 (1987)). Examples of suitable non-radioactive isotopic labels include ¹⁵⁷Gd, ⁵⁵Mn, ¹⁶²Dy, ⁵²Tr, and ⁵⁶Fe.

Examples of suitable fluorescent labels include an ¹⁵²Eu label, a fluorescein label, an isothiocyanate label, a rhodamine label, a phycoerythrin label, a phycocyanin label, an allophycocyanin label, a Green Fluorescent Protein (GFP) label, an o-phthaldehyde label, and a fluorescamine label. Examples of suitable toxin labels include diphtheria toxin, ricin, and cholera toxin.

Examples of chemiluminescent labels include a luminol label, an isoluminol label, an aromatic acridinium ester label, an imidazole label, an acridinium salt label, an oxalate ester label, a luciferin label, a luciferase label, and an aequorin label. Examples of nuclear magnetic resonance contrasting agents include heavy metal nuclei such as Gd, Mn, and iron.

The detection method of the present technology can be used to detect an immunoreactive ASIC1a protein in a biological sample in vitro as well as in vivo. In vitro techniques for detection of an immunoreactive ASIC1a protein include enzyme linked immunosorbent assays (ELISAs), Western blots, immunoprecipitations, radioimmunoassay, and immunofluorescence. Furthermore, in vivo techniques for detection of an immunoreactive ASIC1a protein include introducing into a subject a labeled the anti-ASIC1a antibodies of the present technology antibody. For example, the anti-ASIC1a antibodies of the present technology antibody can be labeled with a radioactive marker whose presence and location in a subject can be detected by standard imaging techniques. In one embodiment, the biological sample contains ASIC1a protein molecules from the test subject.

Immunoassay and Imaging. The anti-ASIC1a antibodies of the present technology can be used to assay immunoreactive ASIC1a protein levels in a biological sample (e.g., human plasma) using antibody-based techniques. For example, protein expression in tissues can be studied with classical immunohistological methods. Jalkanen, M. et al., J. Cell. Biol. 101: 976-985, 1985; Jalkanen, M. et al., J. Cell. Biol. 105: 3087-3096, 1987. Other antibody-based methods useful for detecting protein gene expression include immunoassays, such as the enzyme linked immunosorbent assay (ELISA) and the radioimmunoassay (RIA). Suitable antibody assay labels are known in the art and include enzyme labels, such as, glucose oxidase, and radioisotopes or other radioactive agent, such as iodine (¹²⁵I, ¹²¹I, ¹³¹I) carbon (¹⁴C), sulfur (³⁵S), tritium (³H), indium (¹¹²In), and technetium (⁹⁹mTc), and fluorescent labels, such as fluorescein, rhodamine, and green fluorescent protein (GFP), as well as biotin.

In addition to assaying immunoreactive ASIC1a protein levels in a biological sample, the anti-ASIC1a antibodies of the present technology may be used for in vivo imaging of ASIC1a protein. Antibodies useful for this method include those detectable by X-radiography, NMR or ESR. For X-radiography, suitable labels include radioisotopes such as barium or cesium, which emit detectable radiation but are not overtly harmful to the subject. Suitable markers for NMR and ESR include those with a detectable characteristic spin, such as deuterium, which can be incorporated into the anti-ASIC1a antibodies of the present technology antibodies by labeling of nutrients for the relevant scFv clone.

An anti-ASIC1a antibodies of the present technology which has been labeled with an appropriate detectable imaging moiety, such as a radioisotope (e.g., ¹³¹I, ¹¹²I, ⁹⁹mTc), a radio-opaque substance, or a material detectable by nuclear magnetic resonance, is introduced (e.g., parenterally, subcutaneously, or intraperitoneally) into the subject. It will be understood in the art that the size of the subject and the imaging system used will determine the quantity of imaging moiety needed to produce diagnostic images. In the case of a radioisotope moiety, for a human subject, the quantity of radioactivity injected will normally range from about 5 to 20 millicuries of ⁹⁹mTc. The labeled the anti-ASIC1a antibodies of the present technology antibody will then accumulate at the location of cells which contain the specific target polypeptide. For example, labeled the anti-ASIC1a antibodies of the present technology will accumulate within the subject in cells and tissues in which the ASIC1a protein has localized.

Thus, the present technology provides a diagnostic method of a medical condition, which involves: (a) assaying the expression of immunoreactive ASIC1a protein by measuring binding of the anti-ASIC1a antibodies of the present technology in cells or body fluid of an individual; (b) comparing the amount of immunoreactive ASIC1a protein present in the sample with a standard reference, wherein an increase or decrease in immunoreactive ASIC1a protein levels compared to the standard is indicative of a medical condition.

Affinity Purification. The anti-ASIC1a antibodies of the present technology may be used to purify immunoreactive ASIC1a protein from a sample. In some embodiments, the antibodies are immobilized on a solid support. Examples of such solid supports include plastics such as polycarbonate, complex carbohydrates such as agarose and sepharose, acrylic resins and such as polyacrylamide and latex beads. Techniques for coupling antibodies to such solid supports are well known in the art (Weir et al., “Handbook of Experimental Immunology” 4th Ed., Blackwell Scientific Publications, Oxford, England, Chapter 10 (1986); Jacoby et al., Meth. Enzym. 34 Academic Press, N.Y. (1974)).

The simplest method to bind the antigen to the antibody-support matrix is to collect the beads in a column and pass the antigen solution down the column. The efficiency of this method depends on the contact time between the immobilized antibody and the antigen, which can be extended by using low flow rates. The immobilized antibody captures the antigen as it flows past. Alternatively, an antigen can be contacted with the antibody-support matrix by mixing the antigen solution with the support (e.g., beads) and rotating or rocking the slurry, allowing maximum contact between the antigen and the immobilized antibody. After the binding reaction has been completed, the slurry is passed into a column for collection of the beads. The beads are washed using a suitable washing buffer and then the pure or substantially pure antigen is eluted.

An antibody or polypeptide of interest can be conjugated to a solid support, such as a bead. In addition, a first solid support such as a bead can also be conjugated, if desired, to a second solid support, which can be a second bead or other support, by any suitable means, including those disclosed herein for conjugation of a polypeptide to a support. Accordingly, any of the conjugation methods and means disclosed herein with reference to conjugation of a polypeptide to a solid support can also be applied for conjugation of a first support to a second support, where the first and second solid support can be the same or different.

Appropriate linkers, which can be cross-linking agents, for use for conjugating a polypeptide to a solid support include a variety of agents that can react with a functional group present on a surface of the support, or with the polypeptide, or both. Reagents useful as cross-linking agents include homo-bi-functional and, in particular, hetero-bi-functional reagents. Useful bi-functional cross-linking agents include, but are not limited to, N-SIAB, dimaleimide, DTNB, N-SATA, N-SPDP, SMCC and 6-HYNIC. A cross-linking agent can be selected to provide a selectively cleavable bond between a polypeptide and the solid support. For example, a photolabile cross-linker, such as 3-amino-(2-nitrophenyl)propionic acid can be employed as a means for cleaving a polypeptide from a solid support. (Brown et al., Mol. Divers, pp, 4-12 (1995); Rothschild et al., Nucl. Acids Res., 24:351-66 (1996); and U.S. Pat. No. 5,643,722). Other cross-linking reagents are well-known in the art. (See, e.g., Wong (1991), supra; and Hermanson (1996), supra).

An antibody or polypeptide can be immobilized on a solid support, such as a bead, through a covalent amide bond formed between a carboxyl group functionalized bead and the amino terminus of the polypeptide or, conversely, through a covalent amide bond formed between an amino group functionalized bead and the carboxyl terminus of the polypeptide. In addition, a bi-functional trityl linker can be attached to the support, e.g., to the 4-nitrophenyl active ester on a resin, such as a Wang resin, through an amino group or a carboxyl group on the resin via an amino resin. Using a bi-functional trityl approach, the solid support can require treatment with a volatile acid, such as formic acid or trifluoroacetic acid to ensure that the polypeptide is cleaved and can be removed. In such a case, the polypeptide can be deposited as a beadless patch at the bottom of a well of a solid support or on the flat surface of a solid support. After addition of a matrix solution, the polypeptide can be desorbed into a MS.

Hydrophobic trityl linkers can also be exploited as acid-labile linkers by using a volatile acid or an appropriate matrix solution, e.g., a matrix solution containing 3-HPA, to cleave an amino linked trityl group from the polypeptide. Acid lability can also be changed. For example, trityl, monomethoxytrityl, dimethoxytrityl or trimethoxytrityl can be changed to the appropriate p-substituted, or more acid-labile tritylamine derivatives, of the polypeptide, i.e., trityl ether and tritylamine bonds can be made to the polypeptide. Accordingly, a polypeptide can be removed from a hydrophobic linker, e.g., by disrupting the hydrophobic attraction or by cleaving tritylether or tritylamine bonds under acidic conditions, including, if desired, under typical MS conditions, where a matrix, such as 3-HPA acts as an acid.

Orthogonally cleavable linkers can also be useful for binding a first solid support, e.g., a bead to a second solid support, or for binding a polypeptide of interest to a solid support. Using such linkers, a first solid support, e.g., a bead, can be selectively cleaved from a second solid support, without cleaving the polypeptide from the support; the polypeptide then can be cleaved from the bead at a later time. For example, a disulfide linker, which can be cleaved using a reducing agent, such as DTT, can be employed to bind a bead to a second solid support, and an acid cleavable bi-functional trityl group could be used to immobilize a polypeptide to the support. As desired, the linkage of the polypeptide to the solid support can be cleaved first, e.g., leaving the linkage between the first and second support intact. Trityl linkers can provide a covalent or hydrophobic conjugation and, regardless of the nature of the conjugation, the trityl group is readily cleaved in acidic conditions.

For example, a bead can be bound to a second support through a linking group which can be selected to have a length and a chemical nature such that high density binding of the beads to the solid support, or high density binding of the polypeptides to the beads, is promoted. Such a linking group can have, e.g., “tree-like” structure, thereby providing a multiplicity of functional groups per attachment site on a solid support. Examples of such linking group; include polylysine, polyglutamic acid, penta-erythrole and tris-hydroxy-aminomethane.

Noncovalent Binding Association. An antibody or polypeptide can be conjugated to a solid support, or a first solid support can also be conjugated to a second solid support, through a noncovalent interaction. For example, a magnetic bead made of a ferromagnetic material, which is capable of being magnetized, can be attracted to a magnetic solid support, and can be released from the support by removal of the magnetic field. Alternatively, the solid support can be provided with an ionic or hydrophobic moiety, which can allow the interaction of an ionic or hydrophobic moiety, respectively, with a polypeptide, e.g., a polypeptide containing an attached trityl group or with a second solid support having hydrophobic character.

A solid support can also be provided with a member of a specific binding pair and, therefore, can be conjugated to a polypeptide or a second solid support containing a complementary binding moiety. For example, a bead coated with avidin or with streptavidin can be bound to a polypeptide having a biotin moiety incorporated therein, or to a second solid support coated with biotin or derivative of biotin, such as iminobiotin.

It should be recognized that any of the binding members disclosed herein or otherwise known in the art can be reversed. Thus, biotin, e.g., can be incorporated into either a polypeptide or a solid support and, conversely, avidin or other biotin binding moiety would be incorporated into the support or the polypeptide, respectively. Other specific binding pairs contemplated for use herein include, but are not limited to, hormones and their receptors, enzyme, and their substrates, a nucleotide sequence and its complementary sequence, an antibody and the antigen to which it interacts specifically, and other such pairs knows to those skilled in the art.

### A. Diagnostic Uses of the Anti-ASIC1a Antibodies of the Present Technology

General. The anti-ASIC1a antibodies of the present technology are useful in diagnostic methods. As such, the present technology provides methods using the antibodies in the diagnosis of ASIC1a protein activity in a subject. The anti-ASIC1a antibodies of the present technology may be selected such that they have any level of epitope binding specificity and very high binding affinity to a ASIC1a protein. In general, the higher the binding affinity of an antibody the more stringent wash conditions can be performed in an immunoassay to remove nonspecifically bound material without removing target polypeptide. Accordingly, the anti-ASIC1a antibodies of the present technology useful in diagnostic assays usually have binding affinities of about 10⁸ M⁻¹, 10⁹ M⁻¹, 10¹⁰ M⁻¹, 10¹¹ M⁻¹ or 10¹² M⁻¹. Further, it is desirable that the anti-ASIC1a antibodies of the present technology antibodies used as diagnostic reagents have a sufficient kinetic on-rate to reach equilibrium under standard conditions in at least 12 h, at least five (5) h, or at least one (1) hour.

The anti-ASIC1a antibodies of the present technology antibodies can be used to detect an immunoreactive ASIC1a protein in a variety of standard assay formats. Such formats include immunoprecipitation, Western blotting, ELISA, radioimmunoassay, and immunometric assays. See Harlow & Lane, Antibodies, A Laboratory Manual (Cold Spring Harbor Publications, New York, 1988); U.S. Pat. Nos. 3,791,932; 3,839,153; 3,850,752; 3,879,262; 4,034,074, 3,791,932; 3,817,837; 3,839,153; 3,850,752; 3,850,578; 3,853,987; 3,867,517; 3,879,262; 3,901,654; 3,935,074; 3,984,533; 3,996,345; 4,034,074; and 4,098,876. Biological samples can be obtained from any tissue or body fluid of a subject. In certain embodiments, the subject is at an early stage of cancer. In one embodiment, the early stage of cancer is determined by the level or expression pattern of ASIC1a protein in a sample obtained from the subject. In certain embodiments, the sample is selected from the group consisting of urine, blood, serum, plasma, saliva, amniotic fluid, cerebrospinal fluid (CSF), and biopsied body tissue.

Immunometric or sandwich assays are one format for the diagnostic methods of the present technology. See U.S. Pat. Nos. 4,376,110, 4,486,530, 5,914,241, and 5,965,375. Such assays use one antibody, e.g., the anti-ASIC1a antibodies of the present technology antibody or a population of the anti-ASIC1a antibodies of the present technology antibodies immobilized to a solid phase, and another the anti-ASIC1a antibodies of the present technology antibody or a population of the anti-ASIC1a antibodies of the present technology antibodies in solution. Typically, the solution the anti-ASIC1a antibodies of the present technology antibody or population of the anti-ASIC1a antibodies of the present technology antibodies is labeled. If an antibody population is used, the population can contain antibodies binding to different epitope specificities within the target polypeptide. Accordingly, the same population can be used for both solid phase and solution antibody. If the anti-ASIC1a antibodies of the present technology are used, first and second ASIC1a protein monoclonal antibodies having different binding specificities are used for the solid and solution phase. Solid phase (also referred to as “capture”) and solution (also referred to as “detection”) antibodies can be contacted with target antigen in either order or simultaneously. If the solid phase antibody is contacted first, the assay is referred to as being a forward assay. Conversely, if the solution antibody is contacted first, the assay is referred to as being a reverse assay. If the target is contacted with both antibodies simultaneously, the assay is referred to as a simultaneous assay. After contacting the ASIC1a protein with the anti-ASIC1a antibodies of the present technology antibody, a sample is incubated for a period that usually varies from about 10 min to about 24 hr and is usually about 1 hr. A wash step is then performed to remove components of the sample not specifically bound to the anti-ASIC1a antibodies of the present technology antibody being used as a diagnostic reagent. When solid phase and solution antibodies are bound in separate steps, a wash can be performed after either or both binding steps. After washing, binding is quantified, typically by detecting a label linked to the solid phase through binding of labeled solution antibody. Usually for a given pair of antibodies or populations of antibodies and given reaction conditions, a calibration curve is prepared from samples containing known concentrations of target antigen. Concentrations of the immunoreactive ASIC1a protein in samples being tested are then read by interpolation from the calibration curve (i.e., standard curve). Analyte can be measured either from the amount of labeled solution antibody bound at equilibrium or by kinetic measurements of bound labeled solution antibody at a series of time points before equilibrium is reached. The slope of such a curve is a measure of the concentration of the ASIC1a protein in a sample.

Suitable supports for use in the above methods include, e.g., nitrocellulose membranes, nylon membranes, and derivatized nylon membranes, and also particles, such as agarose, a dextran-based gel, dipsticks, particulates, microspheres, magnetic particles, test tubes, microtiter wells, SEPHADEX™ (Amersham Pharmacia Biotech, Piscataway N.J.), and the like. Immobilization can be by absorption or by covalent attachment. Optionally, the anti-ASIC1a antibodies of the present technology antibodies can be joined to a linker molecule, such as biotin for attachment to a surface bound linker, such as avidin.

In some embodiments, the present disclosure provides the anti-ASIC1a antibodies of the present technology conjugated to a diagnostic agent. The diagnostic agent may comprise a radioactive or non-radioactive label, a contrast agent (such as for magnetic resonance imaging, computed tomography or ultrasound), and the radioactive label can be a gamma-, beta-, alpha-, Auger electron-, or positron-emitting isotope. A diagnostic agent is a molecule which is administered conjugated to an antibody moiety, i.e., antibody or antibody fragment, or subfragment, and is useful in diagnosing or detecting a disease by locating the cells containing the antigen.

Useful diagnostic agents include, but are not limited to, radioisotopes, dyes (such as with the biotin-streptavidin complex), contrast agents, fluorescent compounds or molecules and enhancing agents (e.g., paramagnetic ions) for magnetic resonance imaging (MRI). U.S. Pat. No. 6,331,175 describes MRI technique and the preparation of antibodies conjugated to a MRI enhancing agent and is incorporated in its entirety by reference. In some embodiments, the diagnostic agents are selected from the group consisting of radioisotopes, enhancing agents for use in magnetic resonance imaging, and fluorescent compounds. In order to load an antibody component with radioactive metals or paramagnetic ions, it may be necessary to react it with a reagent having a long tail to which are attached a multiplicity of chelating groups for binding the ions. Such a tail can be a polymer such as a polylysine, polysaccharide, or other derivatized or derivatizable chain having pendant groups to which can be bound chelating groups such as, e.g., ethylenediaminetetraacetic acid (EDTA), diethylenetriaminepentaacetic acid (DTPA), porphyrins, polyamines, crown ethers, bis-thiosemicarbazones, polyoximes, and like groups known to be useful for this purpose. Chelates may be coupled to the antibodies of the present technology using standard chemistries. The chelate is normally linked to the antibody by a group which enables formation of a bond to the molecule with minimal loss of immunoreactivity and minimal aggregation and/or internal cross-linking. Other methods and reagents for conjugating chelates to antibodies are disclosed in U.S. Pat. No. 4,824,659. Particularly useful metal-chelate combinations include 2-benzyl-DTPA and its monomethyl and cyclohexyl analogs, used with diagnostic isotopes for radio-imaging. The same chelates, when complexed with non-radioactive metals, such as manganese, iron and gadolinium are useful for MRI, when used along with the ASIC1a protein antibodies of the present technology.

### B. Therapeutic Use of the Anti-ASIC1a Antibodies of the Present Technology

General. In some aspects, the anti-ASIC1a antibodies of the present technology are useful in methods disclosed herein provide therapies for the prevention, amelioration or treatment of ischemic stroke and related conditions.

In some embodiments, the antibody or antigen binding fragment thereof binds ASIC1a protein. In some embodiments, the antibody or antigen binding fragment thereof binds the extracellular domain of the ASIC1a. In some embodiments, the antibody or antigen binding fragment thereof binds an epitope that spans two ASIC1a monomers. In some embodiments, the antibody or antigen binding fragment thereof inhibits the function of ASIC1a trimer.

In one aspect, the present technology relates to a method of treating acidosis in a subject in need thereof, comprising administering a therapeutically effective amount of an effective amount of an antibody or antigen binding fragment thereof comprising a heavy chain immunoglobulin variable domain (V_{H}) and a light chain immunoglobulin variable domain (V_{L}), wherein the V_{H} comprises a V_{H}-CDR1 sequence of SEQ ID NO: 8, a V_{H}—CDR2 sequence of SEQ ID NO: 9, and a V_{H}-CDR3 sequence selected from the group consisting of: SEQ ID NO: 10, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, and SEQ ID NO: 37; and wherein the V_{L} comprises a V_{L}-CDR1 sequence of SEQ ID NO: 3, a V_{L}—CDR2 sequence of SEQ ID NO: 4, and a V_{L}-CDR3 sequence of SEQ ID NO: 5. Additionally, or alternatively, in some embodiments, theV_{H}-CDR3 sequence selected from the group consisting of: SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, and SEQ ID NO: 37.

In one aspect, the present technology relates to a treating ischemic stroke in a subject in need thereof, comprising administering a therapeutically effective amount of an effective amount of an antibody or antigen binding fragment thereof comprising a heavy chain immunoglobulin variable domain (V_{H}) and a light chain immunoglobulin variable domain (V_{L}), wherein the V_{H} comprises a V_{H}-CDR1 sequence of SEQ ID NO: 8, a V_{H}—CDR2 sequence of SEQ ID NO: 9, and a V_{H}-CDR3 sequence selected from the group consisting of: SEQ ID NO: 10, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, and SEQ ID NO: 37; and wherein the V_{L} comprises a V_{L}-CDR1 sequence of SEQ ID NO: 3, a V_{L}—CDR2 sequence of SEQ ID NO: 4, and a V_{L}-CDR3 sequence of SEQ ID NO: 5. Additionally, or alternatively, in some embodiments, theV_{H}-CDR3 sequence selected from the group consisting of: SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, and SEQ ID NO: 37.

In one aspect, the present technology relates to a method of treating a disorder caused by or related to ASIC1a activity and/or signaling in a subject in need thereof, comprising administering a therapeutically effective amount of an effective amount of an antibody or antigen binding fragment thereof a heavy chain immunoglobulin variable domain (V_{H}) and a light chain immunoglobulin variable domain (V_{L}), wherein the V_{H} comprises a V_{H}-CDR1 sequence of SEQ ID NO: 8, a V_{H}—CDR2 sequence of SEQ ID NO: 9, and a V_{H}-CDR3 sequence selected from the group consisting of: SEQ ID NO: 10, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, and SEQ ID NO: 37; and wherein the V_{L} comprises a V_{L}-CDR1 sequence of SEQ ID NO: 3, a V_{L}—CDR2 sequence of SEQ ID NO: 4, and a V_{L}-CDR3 sequence of SEQ ID NO: 5. Additionally, or alternatively, in some embodiments, theV_{H}-CDR3 sequence selected from the group consisting of: SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, and SEQ ID NO: 37.

Additionally, or alternatively, in some embodiments, the antibody, or antigen binding fragment thereof further comprises a Fc domain of an isotype selected from the group consisting of IgG1, IgG2, IgG3, IgG4, IgA1, IgA2, IgM, IgD, and IgE. Additionally, or alternatively, in some embodiments, the antigen binding fragment is selected from the group consisting of Fab, F(ab′)2, Fab′, scFv, and Fv. Additionally, or alternatively, in some embodiments, the antibody is a monoclonal antibody, a chimeric antibody, a humanized antibody, or a bispecific antibody.

Additionally, or alternatively, in some embodiments, the antibody, or antigen binding fragment thereof binds to ASIC1a. Additionally, or alternatively, in some embodiments, the antibody, or antigen binding fragment thereof is an antagonist of ASIC1a. Additionally, or alternatively, in some embodiments, the antibody, or antigen binding fragment thereof inhibits ASIC1a-mediated, acid-induced currents. Additionally, or alternatively, in some embodiments, the antibody, or antigen binding fragment thereof inhibits ASIC1a-mediated, acid-induced calcium influx.

Additionally, or alternatively the VL comprises SEQ ID NO: 2; and the V_{H} comprises: a V_{H}-CDR1 sequence of SEQ ID NO: 8, a V_{H}—CDR2 sequence of SEQ ID NO: 9, and a V_{H}-CDR3 sequence of SEQ ID NO: 11; a V_{H}-CDR1 sequence of SEQ ID NO: 8, a V_{H}-CDR2 sequence of SEQ ID NO: 9, and a V_{H}-CDR3 sequence of SEQ ID NO: 13; a V_{H}-CDR1 sequence of SEQ ID NO: 8, a V_{H}—CDR2 sequence of SEQ ID NO: 9, and a V_{H}-CDR3 sequence of SEQ ID NO: 15; a V_{H}-CDR1 sequence of SEQ ID NO: 8, a V_{H}—CDR2 sequence of SEQ ID NO: 9, and a V_{H}-CDR3 sequence of SEQ ID NO: 17; a V_{H}-CDR1 sequence of SEQ ID NO: 8, a V_{H}—CDR2 sequence of SEQ ID NO: 9, and a V_{H}-CDR3 sequence of SEQ ID NO: 19; a V_{H}-CDR1 sequence of SEQ ID NO: 8, a V_{H}—CDR2 sequence of SEQ ID NO: 9, and a V_{H}-CDR3 sequence of SEQ ID NO: 21; a V_{H}-CDR1 sequence of SEQ ID NO: 8, a V_{H}-CDR2 sequence of SEQ ID NO: 9, and a V_{H}-CDR3 sequence of SEQ ID NO: 23; a V_{H}-CDR1 sequence of SEQ ID NO: 8, a V_{H}—CDR2 sequence of SEQ ID NO: 9, and a V_{H}-CDR3 sequence of SEQ ID NO: 25; a V_{H}-CDR1 sequence of SEQ ID NO: 8, a V_{H}—CDR2 sequence of SEQ ID NO: 9, and a V_{H}-CDR3 sequence of SEQ ID NO: 27; a V_{H}-CDR1 sequence of SEQ ID NO: 8, a V_{H}—CDR2 sequence of SEQ ID NO: 9, and a V_{H}-CDR3 sequence of SEQ ID NO:29; a V_{H}-CDR1 sequence of SEQ ID NO: 8, a V_{H}—CDR2 sequence of SEQ ID NO: 9, and a V_{H}-CDR3 sequence of SEQ ID NO: 31; a V_{H}-CDR1 sequence of SEQ ID NO: 8, a V_{H}-CDR2 sequence of SEQ ID NO: 9, and a V_{H}-CDR3 sequence of SEQ ID NO: 33; a V_{H}-CDR1 sequence of SEQ ID NO: 8, a V_{H}—CDR2 sequence of SEQ ID NO: 9, and a V_{H}-CDR3 sequence of SEQ ID NO: 35; or a V_{H}—CDR1 sequence of SEQ ID NO: 8, a V_{H}—CDR2 sequence of SEQ ID NO: 9, and a V_{H}-CDR3 sequence of SEQ ID NO: 37.

Additionally, or alternatively, in some embodiments, the V_{L} comprises a V_{L}-CDR1 sequence of SEQ ID NO: 3, a V_{L}—CDR2 sequence of SEQ ID NO: 4, a V_{L}—CDR3 sequence of SEQ ID NO: 5, and the V_{H} comprises: a V_{H}-CDR1 sequence of SEQ ID NO: 8, a V_{H}—CDR2 sequence of SEQ ID NO: 9, and a V_{H}-CDR3 sequence of SEQ ID NO: 11; a V_{H}-CDR1 sequence of SEQ ID NO: 8, a V_{H}—CDR2 sequence of SEQ ID NO: 9, and a V_{H}-CDR3 sequence of SEQ ID NO: 13; a V_{H}-CDR1 sequence of SEQ ID NO: 8, a V_{H}—CDR2 sequence of SEQ ID NO: 9, and a V_{H}-CDR3 sequence of SEQ ID NO: 15; a V_{H}-CDR1 sequence of SEQ ID NO: 8, a V_{H}—CDR2 sequence of SEQ ID NO: 9, and a V_{H}-CDR3 sequence of SEQ ID NO: 17; a V_{H}-CDR1 sequence of SEQ ID NO: 8, a V_{H}—CDR2 sequence of SEQ ID NO: 9, and a V_{H}-CDR3 sequence of SEQ ID NO: 19; a V_{H}-CDR1 sequence of SEQ ID NO: 8, a V_{H}-CDR2 sequence of SEQ ID NO: 9, and a V_{H}-CDR3 sequence of SEQ ID NO: 21; a V_{H}-CDR1 sequence of SEQ ID NO: 8, a V_{H}—CDR2 sequence of SEQ ID NO: 9, and a V_{H}-CDR3 sequence of SEQ ID NO: 23; a V_{H}-CDR1 sequence of SEQ ID NO: 8, a V_{H}—CDR2 sequence of SEQ ID NO: 9, and a V_{H}-CDR3 sequence of SEQ ID NO: 25; a V_{H}-CDR1 sequence of SEQ ID NO: 8, a V_{H}—CDR2 sequence of SEQ ID NO: 9, and a V_{H}-CDR3 sequence of SEQ ID NO:27; a V_{H}-CDR1 sequence of SEQ ID NO: 8, a V_{H}—CDR2 sequence of SEQ ID NO: 9, and a V_{H}-CDR3 sequence of SEQ ID NO:29; a V_{H}-CDR1 sequence of SEQ ID NO: 8, a V_{H}-CDR2 sequence of SEQ ID NO: 9, and a V_{H}-CDR3 sequence of SEQ ID NO: 31; a V_{H}-CDR1 sequence of SEQ ID NO: 8, a V_{H}—CDR2 sequence of SEQ ID NO: 9, and a V_{H}-CDR3 sequence of SEQ ID NO:33; a V_{H}-CDR1 sequence of SEQ ID NO: 8, a V_{H}—CDR2 sequence of SEQ ID NO: 9, and a V_{H}-CDR3 sequence of SEQ ID NO: 35; or a V_{H}—CDR1 sequence of SEQ ID NO: 8, a V_{H}—CDR2 sequence of SEQ ID NO: 9, and a V_{H}-CDR3 sequence of SEQ ID NO: 37.

In some embodiments, the one or more symptoms of ischemic stroke is sudden weakness; paralysis or numbness of the face, arms, or legs, especially on one side of the body; drooping of one side of the face; confusion; difficulty with speaking, such as slurred words, or difficulty understanding speech; trouble seeing in one or both eyes, such as blurred or blackened vision, or double vision in one or both eyes; problems with breathing; dizziness; difficulty with walking; loss of balance or coordination, causing, e.g., unexplained falls; loss of consciousness, and sudden or severe headache. In some embodiments, the one or more symptoms of ischemic stroke is selected from the group consisting of sudden-onset face weakness (such as drooping of one side of the face), arm drift and abnormal speech.

In some embodiments, the antibody or antigen binding fragment thereof is an antibody, scFv, (scFv)₂, Fab, Fab′, F(ab′)₂ or an scFv-Fc antibody. In some embodiments, the antibody or antigen binding fragment thereof is an scFv antibody. In some embodiments, the scFv antibody is ASC06, ASC06-01 ASC06-02 ASC06-03 ASC06-04 ASC06-05 ASC06-06 ASC06-07 ASC06-08 ASC06-09 ASC06-10 ASC06-11 ASC06-12 ASC06-13 or ASC06-14.

Thus, for example, one or more the anti-ASIC1a antibodies of the present technology may be: (1) co-formulated and administered or delivered alone or simultaneously in a combined formulation with other active agents or the anti-ASIC1a antibodies of the present technology; (2) delivered by alternation or in parallel as separate formulations; or (3) by any other combination therapy regimen known in the art. When delivered in alternation therapy, the methods described herein may comprise administering or delivering the active ingredients sequentially, e.g., in separate solution, emulsion, suspension, tablets, pills or capsules, or by different injections in separate syringes. In general, during alternation therapy, an effective dosage of each active ingredient is administered sequentially, i.e., serially, whereas in simultaneous therapy, effective dosages of two or more active ingredients are administered together. Various sequences of intermittent combination therapy may also be used. Administering such combinations of the anti-ASIC1a antibodies of the present technology and other active agents can result in synergistic biological effect when administered in a therapeutically effective amount to a subject suffering from a medical disease or condition and in need of treatment. An advantage of such an approach is that lower doses of the anti-ASIC1a antibodies of the present technology and/or other active agents may be needed to prevent, ameliorate or treat a subject suffering from, or predisposed to, ischemic stroke in a subject. Further, potential side-effects of treatment may be avoided by use of lower dosages of the anti-ASIC1a antibodies of the present technology and/or other active agents.

The anti-ASIC1a antibodies of the present technology are described herein such as ASC06, ASC06-01 ASC06-02 ASC06-03 ASC06-04 ASC06-05 ASC06-06 ASC06-07 ASC06-08 ASC06-09 ASC06-10 ASC06-11 ASC06-12 ASC06-13, ASC06-14, etc. are useful to prevent or treat disease. Specifically, the disclosure provides for both prophylactic and therapeutic methods of treating a subject suffering from, or predisposed to, ischemic stroke. Accordingly, the present methods provide for the prevention and/or treatment a subject suffering from, or predisposed to, ischemic stroke in a subject by administering an effective amount of the anti-ASIC1a antibodies of the present technology to a subject in need thereof to restore of the function of the mutant the ion channel protein trimer. The present technology relates to the treatment of a subject suffering from, or predisposed to, ischemic stroke in mammals through administration of therapeutically effective amounts of the anti-ASIC1a antibodies of the present technology as disclosed herein, such as ASC06, ASC06-01 ASC06-02 ASC06-03 ASC06-04 ASC06-05 ASC06-06 ASC06-07 ASC06-08 ASC06-09 ASC06-10 ASC06-11 ASC06-12 ASC06-13, ASC06-14, etc. to subjects in need thereof.

### Determination of the Biological Effect of the Anti-ASIC1a Antibodies of the Present Technology

In various embodiments, suitable in vitro or in vivo assays are performed to determine the effect of a specific therapeutic based on the anti-ASIC1a antibodies of the present technology and whether its administration is indicated for treatment. In various embodiments, in vitro assays can be performed with representative cell lines, CHO-K1 cells, such as the CHO-K1/hASIC1a (a stable cell line overexpressing the full-length hASIC1a) disclosed herein. These experiments may be used to determine if a given anti-ASIC1a antibodies of the present technology exerts the desired effect in inhibiting the activity of ASIC1a protein trimers. Compounds for use in therapy can be tested in suitable animal model systems including, but not limited to rats, mice, chicken, cows, monkeys, rabbits, and the like, prior to testing in human subjects. Similarly, for in vivo testing, any of the animal model system known in the art can be used prior to administration to human subjects.

In some embodiments, the ASIC1a activity is determined by assays well known in the art, including, but not limited to electrophysiological assays such as patch clamp, as disclosed herein. In some embodiments, the ASIC1a activity is determined by assays that measure biological activity in animal models. In some embodiments, the ASIC1a activity is determined by assays that measure the rescue of disease phenotype of the animal models, including, but not limited to the mouse middle cerebral artery occlusion (MCAO)-induced ischemic stroke model, disclosed herein.

### Modes of Administration and Effective Dosages

Any method known to those in the art for contacting a cell, organ or tissue with a peptide may be employed. Suitable methods include in vitro, ex vivo, or in vivomethods. In vivomethods typically include the administration of an immunoglobulin-related composition, such as those described above, to a mammal, suitably a human. When used in vivo for therapy, the anti-ASIC1a antibodies of the present technology are administered to the subject in effective amounts (i.e., amounts that have desired therapeutic effect). The dose and dosage regimen will depend upon the degree of the symptoms in the subject, the characteristics of the particular immunoglobulin used, e.g., its therapeutic index, the subject, and the subject's history.

The effective amount may be determined during pre-clinical trials and clinical trials by methods familiar to physicians and clinicians. An effective amount of an immunoglobulin useful in the methods may be administered to a mammal in need thereof by any of a number of well-known methods for administering pharmaceutical compounds. The immunoglobulin may be administered systemically or locally.

### C. Kits

The present technology provides kits for the detection and/or treatment of ischemic stroke, comprising at least one immunoglobulin-related composition of the present technology (e.g., any antibody or antigen binding fragment described herein), or a functional variant (e.g., substitutional variant) thereof. Optionally, the above described components of the kits of the present technology are packed in suitable containers and labeled for diagnosis and/or treatment of ischemic stroke, or a disease associated with altered ASIC1 activity or signaling such as neurodegenerative disease, neuropsychological disease, epilepsy, multiple sclerosis, pain and migraine. The above-mentioned components may be stored in unit or multi-dose containers, for example, sealed ampoules, vials, bottles, syringes, and test tubes, as an aqueous, preferably sterile, solution or as a lyophilized, preferably sterile, formulation for reconstitution. The kit may further comprise a second container which holds a diluent suitable for diluting the pharmaceutical composition towards a higher volume. Suitable diluents include, but are not limited to, the pharmaceutically acceptable excipient of the pharmaceutical composition and a saline solution. Furthermore, the kit may comprise instructions for diluting the pharmaceutical composition and/or instructions for administering the pharmaceutical composition, whether diluted or not. The containers may be formed from a variety of materials such as glass or plastic and may have a sterile access port (for example, the container may be an intravenous solution bag or a vial having a stopper which may be pierced by a hypodermic injection needle). The kit may further comprise more containers comprising a pharmaceutically acceptable buffer, such as phosphate-buffered saline, Ringer's solution and dextrose solution. It may further include other materials desirable from a commercial and user standpoint, including other buffers, diluents, filters, needles, syringes, culture medium for one or more of the suitable hosts. The kits may optionally include instructions customarily included in commercial packages of therapeutic or diagnostic products, that contain information about, for example, the indications, usage, dosage, manufacture, administration, contraindications and/or warnings concerning the use of such therapeutic or diagnostic products.

The kits are useful for detecting the presence of an immunoreactive ASIC1a protein in a biological sample, e.g., any body fluid including, but not limited to, e.g., serum, plasma, lymph, cystic fluid, urine, stool, cerebrospinal fluid, ascitic fluid or blood and including biopsy samples of body tissue. For example, the kit can comprise: one or more humanized, chimeric, or bispecific anti-ASIC1a antibodies of the present technology (or antigen binding fragments thereof) capable of binding a ASIC1a protein in a biological sample; means for determining the amount of the ASIC1a protein in the sample; and means for comparing the amount of the immunoreactive ASIC1a protein in the sample with a standard. One or more of the anti-ASIC1a antibodies may be labeled. The kit components, (e.g., reagents) can be packaged in a suitable container. The kit can further comprise instructions for using the kit to detect the immunoreactive ASIC1a protein.

For antibody-based kits, the kit can comprise, e.g., 1) a first antibody, e.g. a humanized, or chimeric anti-ASIC1a antibody of the present technology (or an antigen binding fragment thereof), attached to a solid support, which binds to a ASIC1a protein; and, optionally; 2) a second, different antibody which binds to either the ASIC1a protein or to the first antibody, and is conjugated to a detectable label.

The kit can also comprise, e.g., a buffering agent, a preservative or a protein-stabilizing agent. The kit can further comprise components necessary for detecting the detectable-label, e.g., an enzyme or a substrate. The kit can also contain a control sample or a series of control samples, which can be assayed and compared to the test sample. Each component of the kit can be enclosed within an individual container and all of the various containers can be within a single package, along with instructions for interpreting the results of the assays performed using the kit. The kits of the present technology may contain a written product on or in the kit container. The written product describes how to use the reagents contained in the kit, e.g., for detection of a ASIC1a protein in vitro or in vivo, or for treatment of ischemic stroke in a subject in need thereof. In certain embodiments, the use of the reagents can be according to the methods of the present technology.

### EXAMPLES

The present technology is further illustrated by the following examples, which should not be construed as limiting in any way. For each of the examples below, any immunologic binding agent, such as IgG, IgM, IgA, IgD, IgE, and genetically modified IgG, and fragments thereof described herein could be used. By way of example, but not by limitation, the scFv or IgG1 antibodies used in the examples below could be ASC06, ASC06-01 ASC06-02 ASC06-03 ASC06-04 ASC06-05 ASC06-06 ASC06-07 ASC06-08 ASC06-09 ASC06-10 ASC06-11 ASC06-12 ASC06-13 or ASC06-14, etc.

### Example 1: Affinity Maturation of ASC06 Antibody

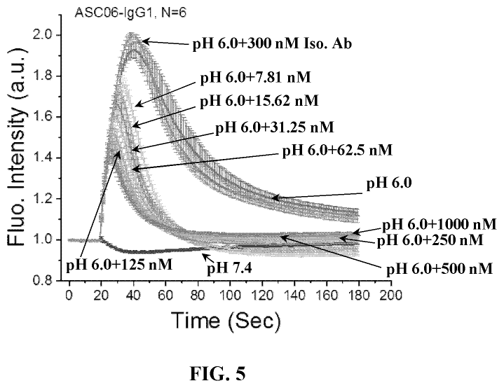

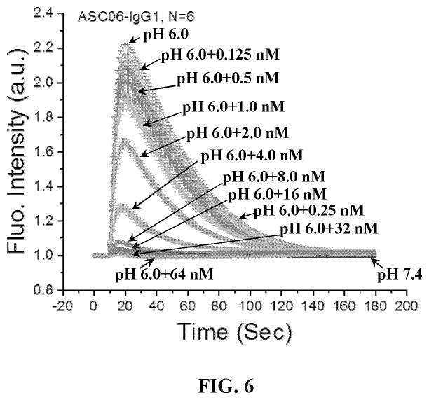

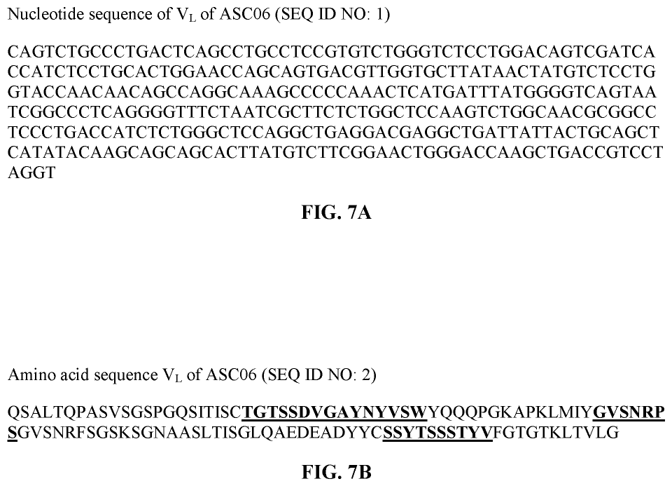

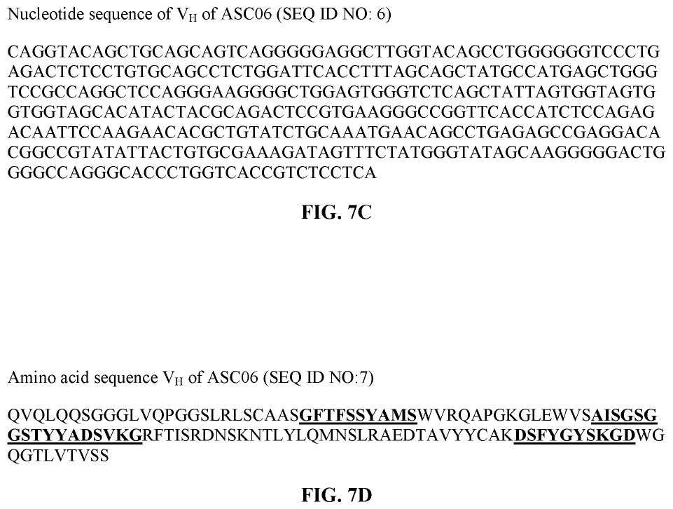

The ASC06 is an Acid-sensing ion channel 1a (ASIC1a)—specific antibody, having antagonist activity against ASIC1a. The Table below, and FIGS. 7A-D provides nucleotide and amino acid of V_{L}, V_{H}, and CDR sequences of ASC06 (SEQ ID NOs: 1-10):

| SEQ ID NO: | Description | Sequence |
|---|---|---|
| SEQ ID NO: 1 | Nucleotide | CAGTCTGCCCTGACTCAGCCTGCCTCCGTGTCTG |
| | sequence of | GGTCTCCTGGACAGTCGATCACCATCTCCTGCAC |
| | V_{L} of ASC06 | TGGAACCAGCAGTGACGTTGGTGCTTATAACTAT |
| | | GTCTCCTGGTACCAACAACAGCCAGGCAAAGCCC |
| | | CCAAACTCATGATTTATGGGGTCAGTAATCGGCC |
| | | CTCAGGGGTTTCTAATCGCTTCTCTGGCTCCAAG |
| | | TCTGGCAACGCGGCCTCCCTGACCATCTCTGGGC |
| | | TCCAGGCTGAGGACGAGGCTGATTATTACTGCAG |
| | | CTCATATACAAGCAGCAGCACTTATGTCTTCGGA |
| | | ACTGGGACCAAGCTGACCGTCCTAGGT |
| SEQ ID NO: 2 | Amino acid | QSALTQPASVSGSPGQSITISCTGTSSDVGAYNY |
| | sequence V_{L} | VSWYQQQPGKAPKLMIYGVSNRPSGVSNRFSGSK |
| | of ASC06 | SGNAASLTISGLQAEDEADYYCSSYTSSSTYVFG |
| | | TGTKLTVLG |
| SEQ ID NO: 3 | V_{L}-CDR1 | TGTSSDVGAYNYVSW |
| SEQ ID NO: 4 | V_{L}-CDR2 | GVSNRPS |
| SEQ ID NO: 5 | V_{L}-CDR3 | SSYTSSSTYV |
| SEQ ID NO: 6 | Nucleotide | CAGGTACAGCTGCAGCAGTCAGGGGGAGGCTTGG |
| | sequence of | TACAGCCTGGGGGGTCCCTGAGACTCTCCTGTGC |
| | V_{H} of ASC06 | AGCCTCTGGATTCACCTTTAGCAGCTATGCCATG |
| | | AGCTGGGTCCGCCAGGCTCCAGGGAAGGGGCTGG |
| | | AGTGGGTCTCAGCTATTAGTGGTAGTGGTGGTAG |
| | | CACATACTACGCAGACTCCGTGAAGGGCCGGTTC |
| | | ACCATCTCCAGAGACAATTCCAAGAACACGCTGT |
| | | ATCTGCAAATGAACAGCCTGAGAGCCGAGGACAC |
| | | GGCCGTATATTACTGTGCGAAAGATAGTTTCTAT |
| | | GGGTATAGCAAGGGGGACTGGGGCCAGGGCACCC |
| | | TGGTCACCGTCTCCTCA |
| SEQ ID NO:7 | Amino acid | QVQLQQSGGGLVQPGGSLRLSCAASGFTFSSYAM |
| | sequence V_{H} | SWVRQAPGKGLEWVSAISGSGGSTYYADSVKGRF |
| | of ASC06 | TISRDNSKNTLYLQMNSLRAEDTAVYYCAKDSFY |
| | | GYSKGDWGQGTLVTVSS |
| SEQ ID NO: 8 | V_{H}-CDR1 | GFTFSSYAMS |
| SEQ ID NO: 9 | V_{H}-CDR2 | AISGSGGSTYYADSVKG |
| SEQ ID NO: 10 | V_{H}-CDR3 | DSFYGYSKGD |

To obtain higher affinity ASIC1a selective antibodies, a mutation sub-library, which comprised mutant V_{H}-CDR3 sequences of the ASC06 antibody, having a diversity of 5×10⁷, was designed and synthesized. The plasmids encoding both heavy chain of ASC06-Fab containing the mutation sub-library and light chain of ASC06-Fab were transfected into yeast competent cells to generate a yeast library using the homologous recombination strategy. To display the ASC06-Fab library on yeast surface, the yeast library was fused with Aga2p protein and EGFP protein.

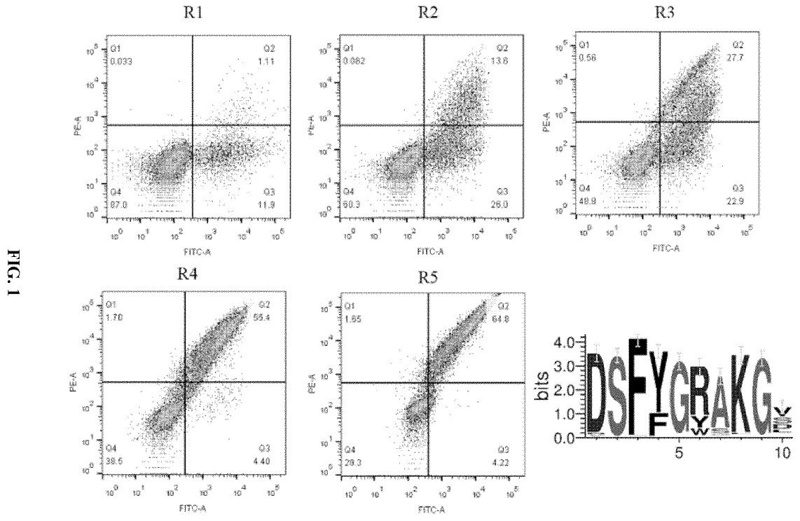

Initially, binding to biotinylated trimeric ectodomain of hASIC1a (hASIC1a-ECD) was used for affinity maturation of the antibody. Subsequently, the yeast library was screened by repeated rounds of fluorescence activated cell sorting (FACS sorting). As shown in FIG. 1, during every round, more specific binders were selected, amplified, and subjected to the next round of FACS sorting. After five rounds of screening, the enriched yeast clones were collected, the plasmids encoding heavy chain were sequenced and analyzed. As shown by the consensus sequence at the bottom right of FIG. 1, the sequence analysis of the plasmids selected after five rounds of selection, including sequence alignment of the V_{H}-CDR3, revealed conservation of certain amino acids within V_{H}-CDR3. Fourteen clones of matured antibodies were identified from the sub-library following the fifth round of screening. The fourteen clones were named as ASC06-01 to ASC06-14. The Table below shows the amino acid sequences of V_{H}-CDR3 of ASC06-01 to ASC06-14, along with the exemplary nucleotide sequences which encode the V_{H}-CDR3 sequences.

| | Amino Acid | Exemplary Nucleotide |
|---|---|---|
| | Sequence of | Sequence of |
| Name | V_{H}-CDR3 | V_{H}-CDR3 |
| ASC06-01 | DSYFGYSKGD | GATAGTTATTTTGGGTATAGCAA |
| | (SEQ ID NO: 11) | GGGGGAC (SEQ ID NO: 12) |
| ASC06-02 | DSFFGRAKGS | GATAGTTTCTTCGGGCGTGCCAA |
| | (SEQ ID NO: 13) | GGGGAGT (SEQ ID NO: 14) |
| ASC06-03 | DSFYGRAKGS | GATAGTTTCTATGGACGCGCAAA |
| | (SEQ ID NO: 15) | GGGGTCT (SEQ ID NO: 16) |
| ASC06-04 | DSFYGRAKGV | GATTCGTTCTATGGGCGTGCAAA |
| | (SEQ ID NO: 17) | GGGGGTC (SEQ ID NO: 18) |
| ASC06-05 | DSYFGRAKGS | GATAGTTACTTCGGGCGTGCCAA |
| | (SEQ ID NO: 19) | GGGGAGT (SEQ ID NO: 20) |
| ASC06-06 | DSFYGRAKGD | GATAGTTTTTATGGGCGGGCGAA |
| | (SEQ ID NO: 21) | AGGGGAC (SEQ ID NO: 22) |
| ASC06-07 | DSFYGYAKGL | GACTCTTTCTATGGGTATGCTAA |
| | (SEQ ID NO: 23) | GGGGCTT (SEQ ID NO: 24) |
| ASC06-08 | DSFFGWAKGV | GATAGTTTCTTCGGGTGGGCTAA |
| | (SEQ ID NO: 25) | GGGGGTA (SEQ ID NO: 26) |
| ASC06-09 | DSFYGRSKGI | GATTCCTTCTATGGGCGCAGCAA |
| | (SEQ ID NO: 27) | GGGGATC (SEQ ID NO: 28) |
| ASC06-10 | DSFYGWAKGL | GATTCGTTCTATGGGTGGGCAAA |
| | (SEQ ID NO: 29) | GGGGCTC (SEQ ID NO: 30) |
| ASCO6-11 | DSFYGRAKGK | GATAGTTTCTATGGGAGAGCAAA |
| | (SEQ ID NO: 31) | GGGGAAA (SEQ ID NO: 32) |
| ASCO6-12 | DSFFGRAKGL | GATAGTTTCTTTGGGCGGGCCAA |
| | (SEQ ID NO: 33) | GGGGTTG (SEQ ID NO: 34) |
| ASC06-13 | VSFFGWAKGD | GTCAGTTTCTTTGGGTGGGCTAA |
| | (SEQ ID NO: 35) | GGGGGAC (SEQ ID NO: 36) |
| ASCO6-14 | DSFFGYAKGH | GATAGTTTCTTTGGGTATGCAAA |
| | (SEQ ID NO: 37) | GGGGCAT (SEQ ID NO: 38) |

The sequences in the Table below, and the consensus sequence shown at the bottom right of FIG. 1 demonstrate that V_{H}-CDR3 may be at least 75, 80, 85, 90 or 95% identical to SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, or SEQ ID NO: 37. These results further demonstrate that using the methods disclosed herein, one may be able to derive antibodies of current disclosure comprising a V_{H}-CDR1 may be at least 75, 80, 85, 90 or 95% identical to the amino acid sequence of SEQ ID NO: 8; a V_{H}-CDR2 may be at least 75, 80, 85, 90 or 95% identical to the amino acid sequence of SEQ ID NO: 9; and a V_{H}-CDR2 may be at least 75, 80, 85, 90 or 95% identical to SEQ ID NO: 9; a V_{H}-CDR3 may be at least 75, 80, 85, 90 or 95% identical to the amino acid sequence of SEQ ID NO: 1; and a V_{L} may be at least 75, 80, 85, 90 or 95% identical to the amino acid sequence of SEQ ID NO: 2.

Five of the fourteen antibodies were further constructed into full length IgG1 format, purified and used for the study of their binding characteristics.

### Example 2: Binding Ability Measurement

Binding affinities of four affinity-matured antibodies in IgG1 format (ASC06-01-IgG1 through ASC06-04-IgG1) to the recombinant extracellular domain of hASIC1a(hASIC1a-ECD) were measured using the Biacore T200™ (GE Healthcare). The ASC06-IgG1 antibody was used as a positive control. All manipulations were followed by the user guide of manufacturer. Briefly, was hASIC1a was immobilized, and serial dilutions of the indicated antibodies were added as analytes. The analysis of the results were processed in BIA Evaluation Software™. The Table below shows the results of the binding measurements. As shown in the Table below, the binding affinity of WT antibody ASC06-IgG1 was 2.8×10⁻¹⁰ M. by comparison, the binding affinities of all the four matured antibodies were higher than that of ASC06-IgG1.

| Antibody | Kₐ (1/Ms) | K_{d} (1/s) | K_{D} (M) |
|---|---|---|---|
| ASC06-IgG1 | 4.7 × 10⁶ | 0.001300 | 2.8 × 10⁻¹⁰ |
| ASC06-01-IgG1 | 9.7 × 10⁶ | 0.000997 | 1.0 × 10⁻¹⁰ |
| ASC06-02-IgG1 | 1.3 × 10⁷ | 0.000168 | 1.25 × 10⁻¹¹ |
| ASC06-03-IgG1 | 6.8 × 10⁵ | 0.000082 | 1.2 × 10⁻¹⁰ |
| ASC06-04-IgG1 | 6.2 × 10⁵ | 0.000163 | 2.6 × 10⁻¹⁰ |

### Example 3: Binding Selectivity

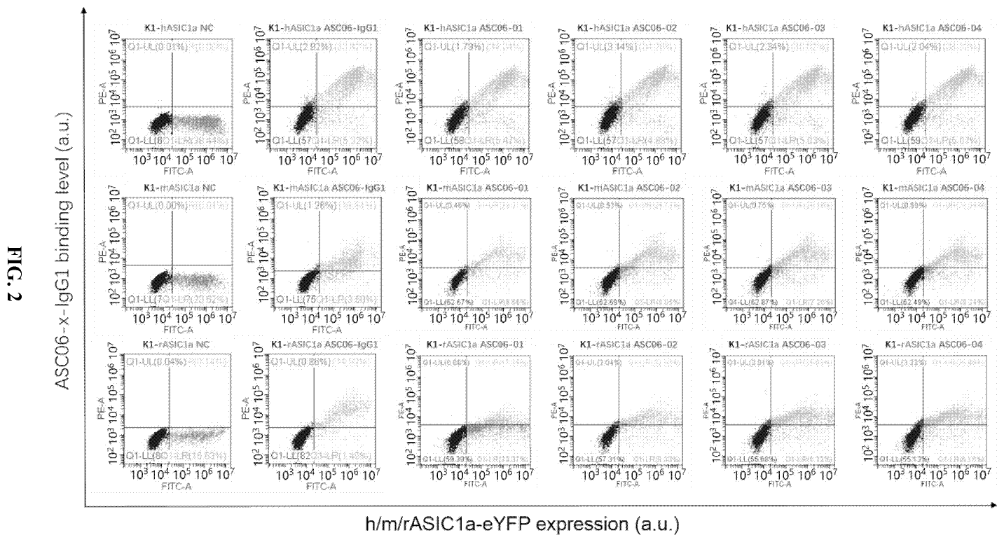

To investigate the species selectivity of ASC06-IgG1 affinity-matured antibodies, plasmids encoding fusion proteins of eYFP with rodent homologs of ASIC1a were constructed. These plasmids included those encoding human ASIC1a (hASIC1a-eYFP), mouse ASIC1a (mASIC1a-eYFP), and rat ASIC1a (rASIC1a-eYFP). CHO-K1 cells were transiently transfected with the ASIC1a-eYFP plasmids, and a fluorescence activated cell sorting (FACS)-based binding assay was carried out to determine binding to the homologs or isoforms of ASIC1a expressed on the cell surface. An isotype control was used as a negative control for binding (NC). ASC06-IgG1 was used as a positive control. Summarily, the CHO-K1 cells expressing the ASIC1a-eYFP (green) homologs were stained with the indicated antibody (red) and subjected to FACS. ASIC1a-binding was detected based on the presence of cell population that was double positive for ASIC1a expression (eYFP, green) and antibody binding (red), which was seen in the upper right quadrant of the FACS profiles. As shown in FIG. 2, the FACS results revealed that ASC06-01-IgG1, ASC06-02-IgG1, ASC06-03-IgG1 and ASC06-04-IgG1 bound to cells expressing hASIC1a-eYFP, mASIC1a-eYFP, and rASIC1a-eYFP, similar to ASC06-IgG1. These data indicate that ASC06-IgG1 and its affinity matured derivatives bound to human and rodent homologs of ASIC1a.

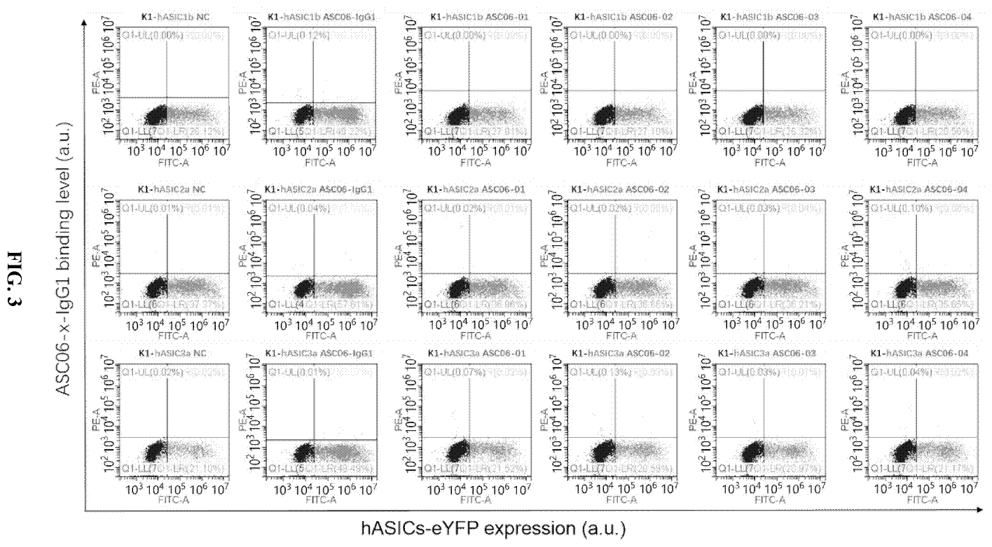

To examine the ASIC isotype selectivity of ASC06-IgG1 affinity-matured antibodies, plasmids encoding fusion proteins of eYFP with isoforms of ASIC1a were constructed. These plasmids included those encoding human ASIC1b (hASIC1b-eYFP), human ASIC2a (hASIC2a-eYFP), and human ASIC3a (hASIC3a-eYFP). CHO-K1 cells were transiently transfected with these plasmids and used in a FACS-based binding assay disclosed herein. Binding of ASC06-IgG1 to CHO-K1 cells expressing human ASIC1a-eYFP (hASIC1a-eYFP) was used as a positive control (data not shown and FIG. 2), and an isotype control was used as a negative control for binding (NC). As shown in FIG. 3, the FACS results showed no double positive cells suggesting that ASC06-IgG1 and its derivatives ASC06-01-IgG1, ASC06-02-IgG1, ASC06-03-IgG1 and ASC06-04-IgG1 showed no detectable binding to the hASIC1b, hASIC2a, or hASIC3a isoforms. These data indicate that ASC06-IgG1 and its affinity matured derivatives bound isotype-specifically to ASIC1a under the assay conditions.

Accordingly, the antibodies or antigen binding fragments of the present technology are useful for methods for detecting ASIC1a in a biological sample.

### Example 4: Inhibition of the ACID-Induced ASIC1a Currents by the Affinity-Matured ASC06-IgG1 Derivative Antibodies

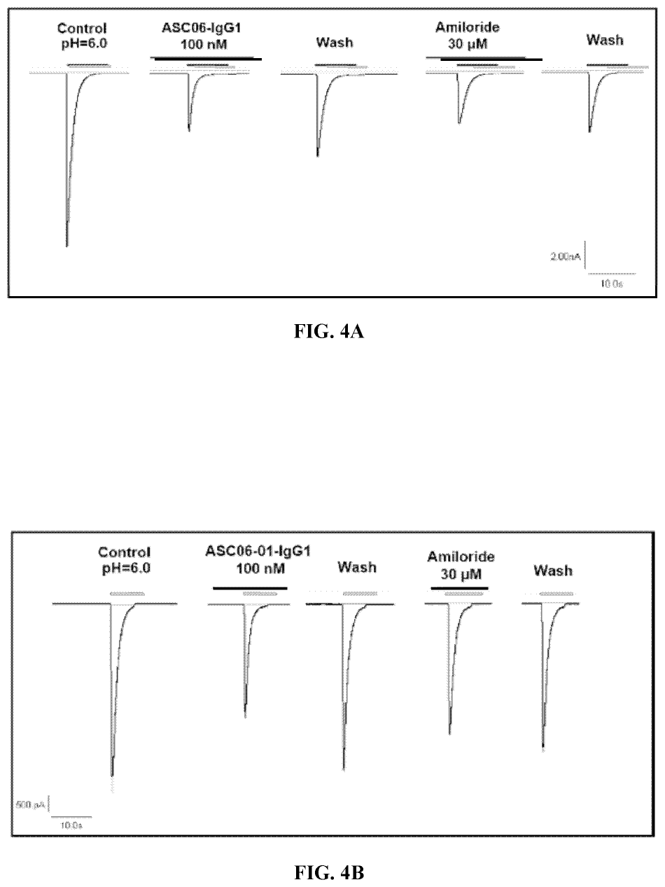

Whether the affinity-matured ASC06-IgG1 derivative antibodies have an effect on the acid-induced, hASIC1a-mediated electrical current in cells was tested. An hASIC1a overexpressing stable cell line was used as a model for these studies. Extracellular pH was decreased from pH 7.4 to pH 6.0 and the amplitudes of the hASIC1a-mediated inward currents were recorded in the whole-cell recording mode in the presence of the affinity-matured ASC06-IgG1 derivative antibodies (FIGS. 4A-4E). The ASIC1a inhibitor amiloride (30 μM) was used as a positive control for the inhibition of ASIC1a currents. As shown in FIG. 4A, decreasing the extracellular pH from pH 7.4 to pH 6.0 resulted in the formation of an electric current in the hASIC1a overexpressing stable cells, and 100 nMASC06-IgG1 displayed >50% inhibition of the acid-induced ASIC1a currents, similar to that observed with 30 μM amiloride (FIG. 4A). In comparison, the same concentration of three of four affinity-matured antibodies (i.e. ASC06-02-IgG1, ASC06-03-IgG1 and ASC06-04-IgG1) showed a stronger blockage of ASIC1a-mediated currents compared to ASC06-IgG1 (FIGS. 4A-4E). The Table below shows the extent of inhibition of the acid induced-hASIC1a currents by ASC06-IgG1 and four affinity-matured derivative antibodies in IgG1 format as measured by Patch Clamp.

| | Percent inhibition of the acid | | |
|---|---|---|---|
| | induced-hASIC1a currents | | |
| Antibody | 30 nM | 100 nM | 300 nM |
| ASC06-IgG1 | 35.5 ± 5.3 | 55.5 ± 10.7 | |
| ASC06-01-IgG1 | 20.0 ± 5.5 | 49.0 ± 8.4 | |
| ASC06-02-IgG1 | 94.6 ± 7.5 | 97.2 ± 2.3 | |
| ASC06-03-IgG1 | 79.4 ± 9.2 | 89.7 ± 5.5 | |
| ASC06-04-IgG1 | | 96.7 ± 1.1 | 96.8 ± 3.4 |

Data are shown as mean ± standard deviation of at least three repeats.

These data demonstrate that ASC06-IgG1 and the affinity-matured derivatives thereof are antagonists of ASIC1a, and are thus useful in methods for treating a subject suffering from, or predisposed to, acidosis, or for treating a subject suffering from a disease caused by or related to increased ASIC1a activity and/or signaling, including ischemic stroke and related conditions.

### Example 5: FLIPR-Based Fluorescent Membrane Potential (FMP) Assay

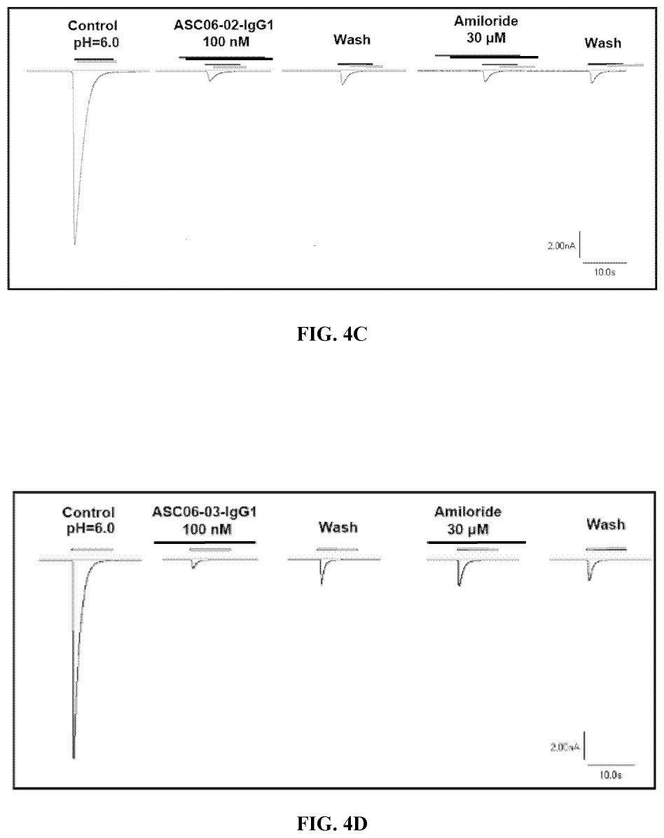

A fluorescence-based assay using the FLIPR Membrane Potential Assay Kit (FMP kit) (Molecular Devices) was used for functional characterization of ASC06-IgG1 and the affinity-matured derivatives thereof. Specifically, the role of ASIC1a in acidosis and the effect of the antibodies of current disclosure was probed further. The FMP dye in the kit is a lipophilic, anionic, bis-oxonol dye, which permits a sensitive evaluation of changes in membrane potential with a more rapid response time. Using the FMP kit, a sensitive cell-based assay to detect acid-induced ASIC1a currents was developed using a cell line stably expressing ASIC1a. The ASIC1a-expressing stable cells were seeded in 96-well plates. The cells were treated with different concentrations of ASC06-IgG1. Untreated cells were used as a negative control. ASIC1a was stimulated by inducing acidosis by decreasing the extracellular pH to 6, and the changes in the fluorescent signal of the FMP dye were measured. An isotype control was used as a negative control for binding. As shown in FIG. 5, ASC06-IgG1 exhibited a dose-dependent inhibition of the fluorescent intensities, which was indicative of the inhibition of ASIC1a-mediated current. The assay was applied to detect the efficiency of inhibition of ASIC1a currents by ASC06-IgG1, and affinity-matured derivatives thereof.

Using similar assays, the IC₅₀ values for inhibition of ASIC1a currents were calculated based on the maximal fluorescent intensity of each concentration of antibodies compared to the negative control. The Table below shows IC₅₀ values for the inhibition of ASIC1a currents by ASC06-IgG1 and affinity-matured derivatives thereof as measured by the FMP assay.

| Antibody | IC₅₀ | |
|---|---|---|
| ASC06-IgG1 | 250.66 | nM |
| ASC06-01-IgG1 | 73.95 | nM |
| ASC06-02-IgG1 | 30.47 | nM |
| ASC06-04-IgG1 | 51.77 | nM |
| ASC06-05-IgG1 | 31.01 | nM |

These data demonstrate that ASC06-01-IgG1 to ASC06-04-IgG1 are more potent antagonists of ASIC1a compared to the parental ASC06-IgG1 antibody.

As discussed above the upregulation of acid-sensing ion channel ASIC1a is associated with the pathogenesis of neurodegenerative disease, neuropsychological disease, epilepsy, multiple sclerosis, pain and migraine, including acidosis. These results demonstrate that ASC06-IgG1 and the affinity-matured derivatives thereof are antagonists of ASIC1a and are thus useful in methods for treating a subject suffering from, or predisposed to, acidosis, or for treating a subject suffering from a disease caused by or related to increased ASIC1a activity and/or signaling, including ischemic stroke and related conditions.

### Example 6: FLIPR-Based Assay to Measure ASIC1a Mediated Calcium Influx

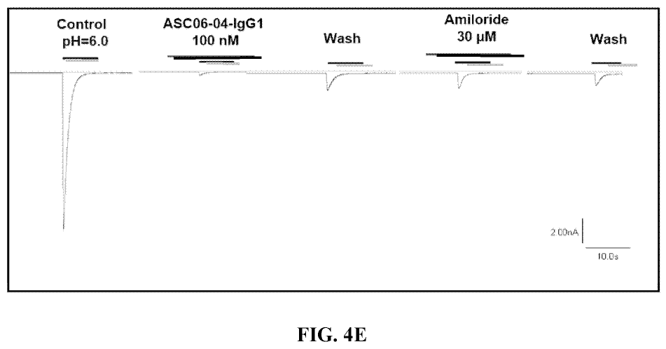

The calcium influx of the ASIC1a channel was measured using a Fluorescence Imaging Plate Reader (FLIPR) instrument by measuring the fluorescent signal generated by the intracellular calcium indicator dye Calcium 5 (Molecular Devices) in a stable cell line expressing hASIC1a-m Cherry fusion. As shown in FIG. 6, the activation of the homomeric hASIC1a channel by decreasing the extracellular pH to 6 induced a strong calcium influx at the tenth second of recording. ASC06-IgG1 displayed a dose-dependent inhibition of calcium influx (FIG. 6).

The inhibition to calcium influx the affinity-matured ASC06-IgG1 derivative antibodies was also measured using the FLIPR-based assay. Using these assays, the IC₅₀ values were calculated based on the maximal fluorescent intensity of the intracellular calcium indicator dye. The Table below shows the IC₅₀ values for the inhibition of acid induced ASIC1a-mediated calcium influx by ASC06-IgG1 and the affinity-matured derivatives thereof as measured by the FLIPR-based assay.

| Antibody | IC50 |
|---|---|
| ASC06-IgG1 | 2.11 nM |
| ASC06-01-IgG1 | 2.24 nM |
| ASC06-02-IgG1 | 1.97 nM |
| ASC06-03-IgG1 | 2.61 nM |

These results demonstrate that ASC06-IgG1 and the affinity-matured derivatives thereof are antagonists of ASIC1a, and are thus useful in methods for treating a subject suffering from, or predisposed to, acidosis, or for treating a subject suffering from a disease caused by or related to increased ASIC1a activity and/or signaling, including ischemic stroke and related conditions.

### Example 7: The Effect of ASC06-IgG1 Derivatives on Acidosis-Induced Cell Death In Vitro

Extracellular acidosis in stroke or ischemia-reperfusion injury is known to induce the activation of ASIC1a channels, which leads to neuronal death in the central nervous system, most likely through transient increase of intracellular calcium and related cell signaling mediated by ASIC1a. The survival of hASIC1a overexpressing stable cells will be assessed upon decreasing the extracellular pH. The pH sensitivity of the control CHO-K1 cells will be compared with that of CHO-K1 cells overexpressing the hASIC1a, especially at pH 5.5. Varying concentrations of ASC06-01-IgG1 to ASC06-14-IgG1 will be added to the cells and a dose-dependent protective effect will be assayed.

These results will demonstrate that the antibodies of the present technology are useful in methods for preventing acidosis-induced cell death, and are thus useful in methods for treating a subject suffering from, or predisposed to, acidosis.

### Example 12: The Effect of ASC06-01-IgG1 to ASC06-14-IgG1 on Acidosis-Induced Cell Death In Vivo

To determine if the protective effect of antibody ASC06-IgG1 in vitro could be extended to pathologies in vivo, the middle cerebral artery occlusion (MCAO) model will be used to study the antibody's neuroprotective effect. Ischemia will be induced by MCAO on the left brain hemisphere of the mice for 60 minutes before reperfusion. Increasing doses of one of more of ASC06-01-IgG1 to ASC06-14-IgG1 will be injected intra-cerebroventricularly (i.c.v.) into the contralateral hemisphere of the mice. An irrelevant antibody (Isotype) with the same concentration will be administrated as a negative control. The infarct volume of the cortex and striatum will be calculated 24 hours after the injection.

These results will demonstrate that the anti-ASIC1a antibodies of the present technology are useful in methods for preventing acidosis-induced cell death and for treating ischemic stroke.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as were apparent to those skilled in the art. Functionally equivalent methods and apparatuses within the scope of the present technology, in addition to those enumerated herein, were apparent to those skilled in the art from the foregoing descriptions. Such modifications and variations are intended to fall within the scope of the appended claims. The present technology is to be limited only by the terms of the appended claims, along with the full scope of equivalents to which such claims are entitled. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As were understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as were understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

Other embodiments are set forth within the following claims.

1. An antibody, or antigen binding fragment thereof comprising a heavy chain immunoglobulin variable domain (V_{H}) and a light chain immunoglobulin variable domain (V_{L}),
wherein the V_{H} comprises a V_{H}-CDR1 sequence of SEQ ID NO: 8, a V_{H}-CDR2 sequence of SEQ ID NO: 9, and a V_{H}-CDR3 sequence selected from the group consisting of: SEQ ID NO: 10, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, and SEQ ID NO: 37; and
wherein the V_{L} comprises a V_{L}-CDR1 sequence of SEQ ID NO: 3, a V_{L}—CDR2 sequence of SEQ ID NO: 4, and a V_{L}-CDR3 sequence of SEQ ID NO: 5.

2. The antibody, or antigen binding fragment thereof of claim 1 further comprising a Fc domain of an isotype selected from the group consisting of IgG1, IgG2, IgG3, IgG4, IgA1, IgA2, IgM, IgD, and IgE.

3. The antibody, or antigen binding fragment thereof of claim 1, wherein the antigen binding fragment is selected from the group consisting of Fab, F(ab′)₂, Fab′, scFᵥ, and Fᵥ.

4. The antibody, or antigen binding fragment thereof of claim 1, wherein the antibody is a monoclonal antibody, a chimeric antibody, a humanized antibody, or a bispecific antibody.

5. The antibody, or antigen binding fragment thereof of claim 1, wherein the antibody, or antigen binding fragment thereof binds to ASIC1a and/or is an antagonist of ASIC1a.

6. (canceled)

7. The antibody, or antigen binding fragment thereof of claim 1, wherein the antibody, or antigen binding fragment thereof inhibits ASIC1a-mediated, acid-induced currents.

8. The antibody, or antigen binding fragment thereof of claim 1, wherein the antibody, or antigen binding fragment thereof inhibits ASIC1a-mediated, acid-induced calcium influx.

9. The antibody, or antigen binding fragment thereof of claim 1, wherein the V_{L} comprises SEQ ID NO: 2; and wherein the V_{H} comprises
a V_{H}-CDR1 sequence of SEQ ID NO: 8, a V_{H}-CDR2 sequence of SEQ ID NO: 9, and a V_{H}-CDR3 sequence of SEQ ID NO: 11;
a V_{H}-CDR1 sequence of SEQ ID NO: 8, a V_{H}-CDR2 sequence of SEQ ID NO: 9, and a V_{H}-CDR3 sequence of SEQ ID NO: 13;
a V_{H}-CDR1 sequence of SEQ ID NO: 8, a V_{H}-CDR2 sequence of SEQ ID NO: 9, and a V_{H}-CDR3 sequence of SEQ ID NO: 15;
a V_{H}-CDR1 sequence of SEQ ID NO: 8, a V_{H}-CDR2 sequence of SEQ ID NO: 9, and a V_{H}-CDR3 sequence of SEQ ID NO: 17;
a V_{H}-CDR1 sequence of SEQ ID NO: 8, a V_{H}-CDR2 sequence of SEQ ID NO: 9, and a V_{H}-CDR3 sequence of SEQ ID NO: 19;
a V_{H}-CDR1 sequence of SEQ ID NO: 8, a V_{H}-CDR2 sequence of SEQ ID NO: 9, and a V_{H}-CDR3 sequence of SEQ ID NO: 21;
a V_{H}-CDR1 sequence of SEQ ID NO: 8, a V_{H}-CDR2 sequence of SEQ ID NO: 9, and a V_{H}-CDR3 sequence of SEQ ID NO: 23;
a V_{H}-CDR1 sequence of SEQ ID NO: 8, a V_{H}-CDR2 sequence of SEQ ID NO: 9, and a V_{H}-CDR3 sequence of SEQ ID NO: 25;
a V_{H}-CDR1 sequence of SEQ ID NO: 8, a V_{H}-CDR2 sequence of SEQ ID NO: 9, and a V_{H}-CDR3 sequence of SEQ ID NO: 27;
a V_{H}-CDR1 sequence of SEQ ID NO: 8, a V_{H}-CDR2 sequence of SEQ ID NO: 9, and a V_{H}-CDR3 sequence of SEQ ID NO:29;
a V_{H}-CDR1 sequence of SEQ ID NO: 8, a V_{H}-CDR2 sequence of SEQ ID NO: 9, and a V_{H}-CDR3 sequence of SEQ ID NO: 31;
a V_{H}-CDR1 sequence of SEQ ID NO: 8, a V_{H}-CDR2 sequence of SEQ ID NO: 9, and a V_{H}-CDR3 sequence of SEQ ID NO: 33;
a V_{H}-CDR1 sequence of SEQ ID NO: 8, a V_{H}-CDR2 sequence of SEQ ID NO: 9, and a V_{H}-CDR3 sequence of SEQ ID NO: 35; or
a V_{H}-CDR1 sequence of SEQ ID NO: 8, a V_{H}-CDR2 sequence of SEQ ID NO: 9, and a V_{H}-CDR3 sequence of SEQ ID NO: 37.

10. An antibody, or antigen binding fragment thereof comprising a heavy chain immunoglobulin variable domain (V_{H}) and a light chain immunoglobulin variable domain (V_{L}), wherein the V_{H} comprises an amino acid sequence of SEQ ID NO:7 and the V_{L} comprises an amino acid sequence of SEQ ID NO: 2.

11. An antibody, or antigen binding fragment thereof, comprising a light chain (LC) and a heavy chain (HC), wherein the LC comprises an amino acid sequence comprising SEQ ID NO: 2, and wherein HC comprises a heavy chain immunoglobulin variable domain (V_{H}), wherein the V_{H} comprises a V_{H}-CDR1 sequence of SEQ ID NO: 8, a V_{H}-CDR2 sequence of SEQ ID NO: 9, and a V_{H}-CDR3 sequence selected from the group consisting of: SEQ ID NO: 10, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, and SEQ ID NO: 37.

12. (canceled)

13. (canceled)

14. A method of treating acidosis in a subject in need thereof, comprising administering a therapeutically effective amount of an effective amount of the antibody or antigen binding fragment of claim 1.

15. A method of treating ischemic stroke in a subject in need thereof, comprising administering a therapeutically effective amount of an effective amount of the antibody or antigen binding fragment of claim 1.

16. A method of treating a disorder caused by or related to ASIC1a activity and/or signaling in a subject in need thereof, comprising administering a therapeutically effective amount of an effective amount of the antibody or antigen binding fragment of claim 1.

17. A nucleic acid sequence encoding the antibody, or antigen binding fragment of claim 1.

18. The nucleic acid sequence of claim 17, wherein the nucleic acid sequence is selected from the group consisting of SEQ ID NOs: 1, 6, 12, 14, 16, 18, 20, 22, 24, 26, 28, 30, 32, 34, 36 and 38.

19. A host cell or a vector expressing the nucleic acid of claim 17.

20. A kit comprising the antibody, or antigen binding fragment thereof of claim 1.

21. The kit of claim 20, wherein the antibody, or antigen binding fragment is coupled to at least one detectable label selected from the group consisting of a radioactive label, a fluorescent label, and a chromogenic label.

22. The kit of claim 20, further comprising a secondary antibody that specifically binds to the antibody, or antigen binding fragment.

23. A method for detecting ASIC1a in a biological sample comprising contacting the biological sample with the antibody, or antigen binding fragment thereof of claim 1, conjugated to a detectable label; and detecting the presence and the level of the detectable label in the biological sample.


SEQUENCE LISTING (38 sequences)

SEQ ID NO: 1 - DNA, 333 bp, Artificial Sequence
  Nucleotide sequence of VL of ASC06
cagtctgccctgactcagcctgcctccgtgtctgggtctcctggacagtcgatcaccatctcctgcactggaaccagcagtgacgttggtgcttataactatgtctcctggtaccaacaacagccaggcaaagcccccaaactcatgatttatggggtcagtaatcggccctcaggggtttctaatcgcttctctggctccaagtctggcaacgcggcctccctgaccatctctgggctccaggctgaggacgaggctgattattactgcagctcatatacaagcagcagcacttatgtcttcggaactgggaccaagctgaccgtcctaggt

SEQ ID NO: 2 - protein, 111 aa, Artificial Sequence
  Amino acid sequence VL of ASC06
QSALTQPASVSGSPGQSITISCTGTSSDVGAYNYVSWYQQQPGKAPKLMIYGVSNRPSGVSNRFSGSKSGNAASLTISGLQAEDEADYYCSSYTSSSTYVFGTGTKLTVLG

SEQ ID NO: 3 - protein, 15 aa, Artificial Sequence
  VL-CDR1
TGTSSDVGAYNYVSW

SEQ ID NO: 4 - protein, 7 aa, Artificial Sequence
  VL-CDR2
GVSNRPS

SEQ ID NO: 5 - protein, 10 aa, Artificial Sequence
  VL-CDR3
SSYTSSSTYV

SEQ ID NO: 6 - DNA, 357 bp, Artificial Sequence
  Nucleotide sequence of VH of ASC06
caggtacagctgcagcagtcagggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaagatagtttctatgggtatagcaagggggactggggccagggcaccctggtcaccgtctcctca

SEQ ID NO: 7 - protein, 119 aa, Artificial Sequence
  Amino acid sequence VH of ASC06
QVQLQQSGGGLVQPGGSLRLSCAASGFTFSSYAMSWVRQAPGKGLEWVSAISGSGGSTYYADSVKGRFTISRDNSKNTLYLQMNSLRAEDTAVYYCAKDSFYGYSKGDWGQGTLVTVSS

SEQ ID NO: 8 - protein, 10 aa, Artificial Sequence
  VH-CDR1
GFTFSSYAMS

SEQ ID NO: 9 - protein, 17 aa, Artificial Sequence
  VH-CDR2
AISGSGGSTYYADSVKG

SEQ ID NO: 10 - protein, 10 aa, Artificial Sequence
  VH-CDR3
DSFYGYSKGD

SEQ ID NO: 11 - protein, 10 aa, Artificial Sequence
  Amino Acid Sequence of VH-CDR3 of ASC06-01
DSYFGYSKGD

SEQ ID NO: 12 - DNA, 30 bp, Artificial Sequence
  Exemplary Nucleotide Sequence of VH-CDR3 of ASC06-01
gatagttattttgggtatagcaagggggac

SEQ ID NO: 13 - protein, 10 aa, Artificial Sequence
  Amino Acid Sequence of VH-CDR3 of ASC06-02
DSFFGRAKGS

SEQ ID NO: 14 - DNA, 30 bp, Artificial Sequence
  Exemplary Nucleotide Sequence of VH-CDR3 of ASC06-02
gatagtttcttcgggcgtgccaaggggagt

SEQ ID NO: 15 - protein, 10 aa, Artificial Sequence
  Amino Acid Sequence of VH-CDR3 of ASC06-03
DSFYGRAKGS

SEQ ID NO: 16 - DNA, 30 bp, Artificial Sequence
  Exemplary Nucleotide Sequence of VH-CDR3 of ASC06-03
gatagtttctatggacgcgcaaaggggtct

SEQ ID NO: 17 - protein, 10 aa, Artificial Sequence
  Amino Acid Sequence of VH-CDR3 of ASC06-04
DSFYGRAKGV

SEQ ID NO: 18 - DNA, 30 bp, Artificial Sequence
  Exemplary Nucleotide Sequence of VH-CDR3 of ASC06-04
gattcgttctatgggcgtgcaaagggggtc

SEQ ID NO: 19 - protein, 10 aa, Artificial Sequence
  Amino Acid Sequence of VH-CDR3 of ASC06-05
DSYFGRAKGS

SEQ ID NO: 20 - DNA, 30 bp, Artificial Sequence
  Exemplary Nucleotide Sequence of VH-CDR3 of ASC06-05
gatagttacttcgggcgtgccaaggggagt

SEQ ID NO: 21 - protein, 10 aa, Artificial Sequence
  Amino Acid Sequence of VH-CDR3 of ASC06-06
DSFYGRAKGD

SEQ ID NO: 22 - DNA, 30 bp, Artificial Sequence
  Exemplary Nucleotide Sequence of VH-CDR3 of ASC06-06
gatagtttttatgggcgggcgaaaggggac

SEQ ID NO: 23 - protein, 10 aa, Artificial Sequence
  Amino Acid Sequence of VH-CDR3 of ASC06-07
DSFYGYAKGL

SEQ ID NO: 24 - DNA, 30 bp, Artificial Sequence
  Exemplary Nucleotide Sequence of VH-CDR3 of ASC06-07
gactctttctatgggtatgctaaggggctt

SEQ ID NO: 25 - protein, 10 aa, Artificial Sequence
  Amino Acid Sequence of VH-CDR3 of ASC06-08
DSFFGWAKGV

SEQ ID NO: 26 - DNA, 30 bp, Artificial Sequence
  Exemplary Nucleotide Sequence of VH-CDR3 of ASC06-08
gatagtttcttcgggtgggctaagggggta

SEQ ID NO: 27 - protein, 10 aa, Artificial Sequence
  Amino Acid Sequence of VH-CDR3 of ASC06-09
DSFYGRSKGI

SEQ ID NO: 28 - DNA, 30 bp, Artificial Sequence
  Exemplary Nucleotide Sequence of VH-CDR3 of ASC06-09
gattccttctatgggcgcagcaaggggatc

SEQ ID NO: 29 - protein, 10 aa, Artificial Sequence
  Amino Acid Sequence of VH-CDR3 of ASC06-10
DSFYGWAKGL

SEQ ID NO: 30 - DNA, 30 bp, Artificial Sequence
  Exemplary Nucleotide Sequence of VH-CDR3 of ASC06-10
gattcgttctatgggtgggcaaaggggctc

SEQ ID NO: 31 - protein, 10 aa, Artificial Sequence
  Amino Acid Sequence of VH-CDR3 of ASC06-11
DSFYGRAKGK

SEQ ID NO: 32 - DNA, 30 bp, Artificial Sequence
  Exemplary Nucleotide Sequence of VH-CDR3 of ASC06-11
gatagtttctatgggagagcaaaggggaaa

SEQ ID NO: 33 - protein, 10 aa, Artificial Sequence
  Amino Acid Sequence of VH-CDR3 of ASC06-12
DSFFGRAKGL

SEQ ID NO: 34 - DNA, 30 bp, Artificial Sequence
  Exemplary Nucleotide Sequence of VH-CDR3 of ASC06-12
gatagtttctttgggcgggccaaggggttg

SEQ ID NO: 35 - protein, 10 aa, Artificial Sequence
  Amino Acid Sequence of VH-CDR3 of ASC06-13
VSFFGWAKGD

SEQ ID NO: 36 - DNA, 30 bp, Artificial Sequence
  Exemplary Nucleotide Sequence of VH-CDR3 of ASC06-13
gtcagtttctttgggtgggctaagggggac

SEQ ID NO: 37 - protein, 10 aa, Artificial Sequence
  Amino Acid Sequence of VH-CDR3 of ASC06-14
DSFFGYAKGH

SEQ ID NO: 38 - DNA, 30 bp, Artificial Sequence
  Exemplary Nucleotide Sequence of VH-CDR3 of ASC06-14
gatagtttctttgggtatgcaaaggggcat